(19) United States
(12) Patent Application Publication
(10) Pub. No.: US 2025/0353830 A1
(43) Pub. Date: Nov. 20, 2025

(54) NOVEL COMPOUNDS

(71) Applicant: Hoffmann-La Roche Inc., Little Falls, NJ (US)

(72) Inventors: Lea Aurelie BOUCHE, Basel (CH); Wolfgang GUBA, Muellheim (DE); Georg JAESCHKE, Basel (CH); Andreas MARX, Tunau (DE); Stefanie Katharina MESCH, Basel (CH); Angélique PATINY-ADAM, Kembs (FR); Christian SCHNIDER, Biel-Benken (CH); Sandra STEINER, Sursee (CH); Andreas Michael TOSSTORFF, Muenchenstein (CH)

(73) Assignee: Hoffmann-La Roche Inc., Little Falls, NJ (US)

(21) Appl. No.: 18/867,741

(22) PCT Filed: Jun. 1, 2023
(86) PCT No.: PCT/EP2023/064634

§ 371 Date: Nov. 20, 2024

(30) Foreign Application Priority Data
Jun. 3, 2022 (EP) 22177110.8
Jun. 3, 2022 (EP) 22177111.6

(51) Int. Cl. C07D 401/14; A61K 31/501; A61K 31/5025; A61K 31/53; C07D 403/04; C07D 471/04; C07D 487/04

(52) U.S. Cl. CPC C07D 401/14; A61K 31/501; A61K 31/5025; A61K 31/53; C07D 403/04; C07D 471/04; C07D 487/04

(57) ABSTRACT

The invention relates to novel compounds having the general formula (Ib) or (Ib′), (Ib) or (Ib′) wherein R¹, R², R⁶, A₃, A₄, A₅, X, Y and W are as described herein, composition including the compounds and methods of using the compounds.

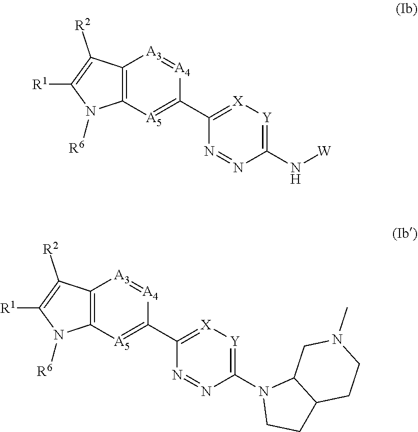

### FIELD OF THE INVENTION

The present invention relates to organic compounds useful for therapy and/or prophylaxis in a mammal, and in particular to compounds that modulate NLRP3 inhibition.

The present invention provides novel compounds of formula Ib or Ib′

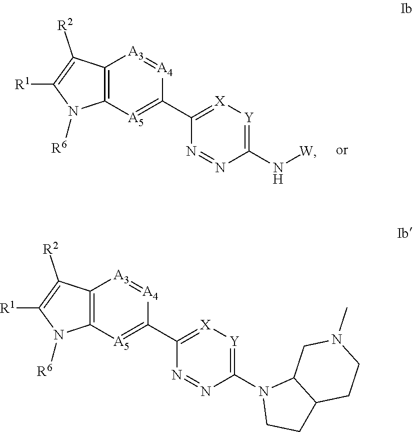

wherein
A₃ is selected from N or CR³;
A₄ is selected from N or CR⁴;
A₅ is selected from N or CR⁵;
Y is selected from N or CH and X is CR⁷,
or X and Y, and the atoms to which they are attached, form a 5-member heteroaryl ring comprising 2 N heteroatoms, wherein one N heteroatom is substituted with either H or alkyl;
R¹ is selected from alkyl, cyano, or H;
R² is selected from alkyl, cyano, H or halo;
R³ is H;
R⁴ is selected from H, alkyl, alkoxy or halo;
R⁵ is selected from H or halo;
R⁶ is H;
R⁷ is alkyl or haloalkyl;
W is optionally substituted cycloalkyl or optionally substituted heterocycle ring comprising a single N heteroatom, wherein optionally substituted cycloalkyl can be substituted with 1 or 2 substituents independently selected from H, OH or alkyl, and optionally substituted heterocycle ring comprising a single N heteroatom can be substituted with alkyl or haloalkyl;
and pharmaceutically acceptable salts thereof.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-la. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-p-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide compounds with improved pharmacological and/or physiological and/or physicochemical properties and/or those that provide a useful alternative to known compounds.

### SUMMARY OF THE INVENTION

The present invention provides novel compounds of formula Ib or Ib′

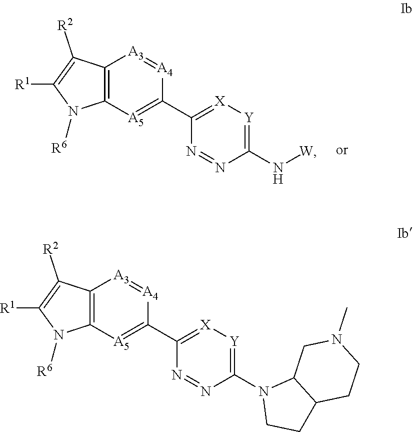

wherein
A₃ is selected from N or CR³;
A₄ is selected from N or CR⁴;
A₅ is selected from N or CR⁵;
Y is selected from N or CH and X is CR⁷,
or X and Y, and the atoms to which they are attached, form a 5-member heteroaryl ring comprising 2 N heteroatoms, wherein one N heteroatom is substituted with either H or alkyl;
R¹ is selected from alkyl, cyano, or H;
R² is selected from alkyl, cyano, H or halo;
R³ is H;
R⁴ is selected from H, alkyl, alkoxy or halo;
R⁵ is selected from H or halo;
R⁶ is H;
R⁷ is alkyl or haloalkyl;
W is optionally substituted cycloalkyl or optionally substituted heterocycle ring comprising a single N heteroatom, wherein optionally substituted cycloalkyl can be substituted with 1 or 2 substituents independently selected from H, OH or alkyl, and optionally substituted heterocycle ring comprising a single N heteroatom can be substituted with alkyl or haloalkyl;
and pharmaceutically acceptable salts thereof.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl and ethyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy.

The term “cyano” denotes a —C≡N group.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. In other embodiments, cycloalkyl comprises one or more double bonds (e.g., cycloalkyl fused to an aryl or heteroaryl ring, or a non-aromatic monocyclic hydrocarbon comprising one or two double bonds). Examples of cycloalkyl groups include cyclopropyl, cyclobutyl, cyclopentyl, cyclohexyl, octahydropentalenyl, spiro[3.3]heptanyl, and the like. Bicyclic means a ring system consisting of two saturated carbocycles having two carbon atoms in common. Examples for monocyclic cycloalkyl are cyclopropyl, cyclobutanyl, cyclopentyl, cyclohexyl or cycloheptyl.

The term “halogen”, “halide” and “halo” are used interchangeably herein and denote fluoro, chloro, bromo or iodo. Particular halogens are fluoro and chloro.

The term “haloalkyl” denotes a C₁₋₆-alkyl group wherein at least one of the hydrogen atoms of the C₁₋₆-alkyl group has been replaced by the same or different halogen atoms. Particular example is trifluoromethyl.

The term “heterocycle” denotes a monovalent saturated or partly unsaturated mono- or bicyclic ring system of 4 to 10 ring atoms, or 4 to 9 ring atoms, comprising 1, 2, or 3 ring heteroatoms selected from N, O and S, the remaining ring atoms being carbon. Examples for monocyclic saturated heterocycle rings are oxetanyl, azetidinyl, pyrrolidinyl, tetrahydrofuranyl, pyrazolidinyl, imidazolidinyl, oxazolidinyl, isoxazolidinyl, thiazolidinyl, piperidinyl, tetrahydropyranyl, tetrahydrothiopyranyl, or piperazinyl. Examples for partly unsaturated heterocycle rings are dihydrofuryl, imidazolinyl, dihydro-oxazolyl, tetrahydro-pyridinyl, or dihydropyranyl. Particular example of a heterocycle ring is piperidinyl.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Examples of heteroaryl group include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridinyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, and benzothiophenyl.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula Ib can also be present in the form of zwitterions. Particularly preferred pharmaceutically acceptable salts of compounds of formula Ib are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula Ib can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula Ib as described herein and pharmaceutically acceptable salts or esters thereof, in particular compounds according to formula Ib as described herein and pharmaceutically acceptable salts thereof, more particularly compounds according to formula Ib as described herein.

Also an embodiment of the present invention provides compounds according to formula Ib′ as described herein and pharmaceutically acceptable salts or esters thereof, in particular compounds according to formula Ib′ as described herein and pharmaceutically acceptable salts thereof, more particularly compounds according to formula Ib′ as described herein.

An embodiment of the present invention provides compounds according to formula Ib as described herein wherein
A₃ is selected from N or CR³;
A₄ is selected from N or CR⁴;
A₅ is selected from N or CR⁵;
Y is selected from N or CH and X is CR⁷,
or X and Y, and the atoms to which they are attached, form a 5-member heteroaryl ring comprising 2 N heteroatoms, wherein one N heteroatom is substituted with either H or alkyl;
R¹ is selected from alkyl, cyano, or H;
R² is selected from alkyl, cyano, H or halo;
R³ is H;
R⁴ is selected from H, alkyl, alkoxy or halo;
R⁵ is selected from H or halo;
R⁶ is H;
R⁷ is alkyl or haloalkyl;
W is optionally substituted cycloalkyl or optionally substituted heterocycle ring comprising a single N heteroatom, wherein optionally substituted cycloalkyl can be substituted with 1 or 2 substituents independently selected from H, OH or alkyl, and optionally substituted heterocycle ring comprising a single N heteroatom can be substituted with alkyl or haloalkyl;
and pharmaceutically acceptable salts thereof.

An embodiment of the present invention provides compounds according to formula Ib as described herein, wherein Y is selected from N or CH and X is CR⁷.

An embodiment of the present invention provides compounds according to formula Ib as described herein, wherein R¹ is H.

An embodiment of the present invention provides compounds according to formula Ib as described herein, wherein R² is H or halo.

An embodiment of the present invention provides compounds according to formula Ib as described herein, wherein A₃ is CR³.

An embodiment of the present invention provides compounds according to formula Ib as described herein, wherein A₄ is CR⁴.

An embodiment of the present invention provides compounds according to formula Ib as described herein, wherein A₅ is CR⁵.

An embodiment of the present invention provides compounds according to formula Ib as described herein, wherein R⁴ is selected from H or halo.

An embodiment of the present invention provides compounds according to formula Ib as described herein, wherein R⁴ is H.

An embodiment of the present invention provides compounds according to formula Ib as described herein, wherein R⁵ is H.

An embodiment of the present invention provides compounds according to formula Ib as described herein, wherein R⁷ is alkyl.

An embodiment of the present invention provides compounds according to formula Ib as described herein, wherein W is a piperidine ring substituted with alkyl.

An embodiment of the present invention provides compounds according to formula Ib, wherein
A₃ is CR³;
A₄ is CR⁴;
A₅ is CR⁵;
Y is selected from N or CH and X is CR⁷;
R¹ is alkyl, cyano, or H;
R² is alkyl, cyano, H or halo;
R³ is H;
R⁴ is H or halo;
R⁵ is H or halo;
R⁶ is H;
R⁷ is alkyl or haloalkyl;
W is a piperidine ring substituted with alkyl;
and pharmaceutically acceptable salts thereof

An embodiment of the present invention provides compounds according to formula Ib, wherein
A₃ is CR³;
A₄ is CR⁴;
A₅ is CR⁵;
Y is selected from N or CH and X is CR⁷;
R¹ is H;
R² is H or halo;
R³ is H;
R⁴ is H;
R⁵ is H;
R⁶ is H;
R⁷ is alkyl or haloalkyl;
W is a piperidine ring substituted with alkyl;
and pharmaceutically acceptable salts thereof.

An embodiment of the present invention provides compounds according to formula Ib, wherein
A₃ is CR³;
A₄ is CR⁴;
A₅ is CR⁵;
Y is selected from N or CH and X is CR⁷;
R¹ is H;
R² is H or halo;
R³ is H;
R⁴ is H;
R⁵ is H;
R⁶ is H;
R⁷ is alkyl;
W is a piperidine ring substituted with alkyl;
and pharmaceutically acceptable salts thereof

Particular examples of compounds of formula Ib as described herein are selected from
N-[(3R)-1-Ethyl-3-piperidyl]-6-(1H-indol-6-yl)-5-methyl-pyridazin-3-amine;
N-[(3R)-1-Ethyl-3-piperidyl]-6-(3-fluoro-1H-indol-6-yl)-5-methyl-pyridazin-3-amine;
N-[(3R)-1-Ethyl-3-piperidyl]-6-(5-fluoro-1H-indol-6-yl)-5-methyl-pyridazin-3-amine;
N-[(3R)-1-Ethyl-3-piperidyl]-5-methyl-6-(3-methyl-1H-indol-6-yl)pyridazin-3-amine;
6-(3-Chloro-1H-indol-6-yl)-N-[(3R)-1-ethyl-3-piperidyl]-5-methyl-pyridazin-3-amine;
N-[(3R)-1-Ethyl-3-piperidyl]-5-methyl-6-(2-methyl-1H-indol-6-yl)pyridazin-3-amine;
6-[6-[[(3R)-1-Ethyl-3-piperidyl]amino]-4-methyl-pyridazin-3-yl]-1H-indole-3-carbonitrile;
6-[6-[[(3R)-1-Ethyl-3-piperidyl]amino]-4-methyl-pyridazin-3-yl]-1H-indole-2-carbonitrile;
6-(3-Fluoro-1H-indol-6-yl)-5-methyl-N-[(3R)-1-methyl-3-piperidyl]pyridazin-3-amine;
N-[(3R)-1-Ethyl-3-piperidyl]-6-(1H-indol-6-yl)-5-methyl-pyridazin-3-amine;
N-[(3R)-1-Ethyl-3-piperidyl]-6-(3-fluoro-1H-indol-6-yl)-5-(trifluoromethyl)pyridazin-3-amine;
5-Ethyl-N-[(3R)-1-ethyl-3-piperidyl]-6-(3-fluoro-1H-indol-6-yl)pyridazin-3-amine;
N-[(3R)-1-Ethyl-3-piperidyl]-6-(1H-indol-6-yl)-5-methyl-1,2,4-triazin-3-amine;
N-[(3R)-1-Ethyl-3-piperidyl]-6-(3-fluoro-1H-indol-6-yl)-5-methyl-1,2,4-triazin-3-amine;
and pharmaceutically acceptable salts thereof.

Other particular examples of formula Ib as described herein are selected from
N-[(3R)-1-Ethyl-3-piperidyl]-6-(5-methyl-1H-indol-6-yl)pyridazin-3-amine;
3-[[6-(3-Fluoro-1H-indol-6-yl)-5-methyl-pyridazin-3-yl]amino]-1-methyl-cyclobutanol;
and pharmaceutically acceptable salts thereof.

An other particular examples of formula Ib as described herein is N-[(3R)-1-Ethyl-3-piperidyl]-7-(1H-indol-6-yl)-1-methyl-pyrazolo[3,4-d]pyridazin-4-amine, and pharmaceutically acceptable salts thereof.

Other particular examples of formula Ib as described herein are selected from
(3aS,7aR)-1-[6-(3-Fluoro-1H-indol-6-yl)-5-methyl-pyridazin-3-yl]-6-methyl-3,3a,4,5,7,7a-hexahydro-2H-pyrrolo[2,3-c]pyridine;
(3aR,7aS)-1-[6-(3-fluoro-1H-indol-6-yl)-5-methyl-pyridazin-3-yl]-6-methyl-3,3a,4,5,7,7a-hexahydro-2H-pyrrolo[2,3-c]pyridine;
and pharmaceutically acceptable salts thereof.

Preferred examples of compounds of formula Ib as described herein are selected from
N-[(3R)-1-Ethyl-3-piperidyl]-6-(1H-indol-6-yl)-5-methyl-pyridazin-3-amine;
N-[(3R)-1-Ethyl-3-piperidyl]-6-(3-fluoro-1H-indol-6-yl)-5-methyl-pyridazin-3-amine;
6-(3-Fluoro-1H-indol-6-yl)-5-methyl-N-[(3R)-1-methyl-3-piperidyl]pyridazin-3-amine;
N-[(3R)-1-Ethyl-3-piperidyl]-6-(3-fluoro-1H-indol-6-yl)-5-(trifluoromethyl)pyridazin-3-amine;
N-[(3R)-1-Ethyl-3-piperidyl]-6-(1H-indol-6-yl)-5-methyl-1,2,4-triazin-3-amine;
N-[(3R)-1-Ethyl-3-piperidyl]-6-(3-fluoro-1H-indol-6-yl)-5-methyl-1,2,4-triazin-3-amine;
and pharmaceutically acceptable salts thereof.

Most preferred examples of compounds of formula Ib as described herein are selected from
N-[(3R)-1-Ethyl-3-piperidyl]-6-(3-fluoro-1H-indol-6-yl)-5-methyl-pyridazin-3-amine;
N-[(3R)-1-Ethyl-3-piperidyl]-6-(1H-indol-6-yl)-5-methyl-1,2,4-triazin-3-amine;
N-[(3R)-1-Ethyl-3-piperidyl]-6-(3-fluoro-1H-indol-6-yl)-5-methyl-1,2,4-triazin-3-amine;
and pharmaceutically acceptable salts thereof.

An embodiment of the present invention provides compounds according to formula I, wherein the compound of formula I is a compound of formula Ib

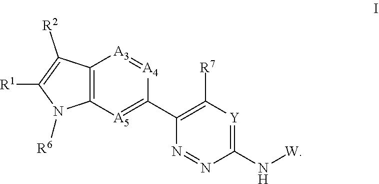

An embodiment of the present invention provides compounds according to formula I as described herein, wherein
A₃ is selected from N or CR³;
A₄ is selected from N or CR⁴;
A₅ is selected from N or CR⁵;
Y is selected from N or CH;
R¹ is selected from alkyl, cyano, or H;
R² is selected from alkyl, cyano, H or halo;
R³ is H;
R⁴ is selected from H, alkyl, alkoxy or halo;
R⁵ is selected from H or halo;
R⁶ is H;
R⁷ is alkyl or haloalkyl;
W is optionally substituted cycloalkyl or optionally substituted heterocycle ring comprising a single N heteroatom, wherein optionally substituted cycloalkyl can be substituted with 1 or 2 substituents independently selected from H, OH or alkyl, and optionally substituted heterocycle ring comprising a single N heteroatom can be substituted with alkyl or haloalkyl;
and pharmaceutically acceptable salts thereof.

An embodiment of the present invention provides compounds according to formula I as described herein, wherein R¹ is H.

An embodiment of the present invention provides compounds according to formula I as described herein, wherein R² is H or halo.

An embodiment of the present invention provides compounds according to formula I as described herein, wherein A₃ is CR³.

An embodiment of the present invention provides compounds according to formula I as described herein, wherein A₄ is CR⁴.

An embodiment of the present invention provides compounds according to formula I as described herein, wherein A₅ is CR⁵.

An embodiment of the present invention provides compounds according to formula I as described herein, wherein R⁴ is selected from H or halo.

An embodiment of the present invention provides compounds according to formula I as described herein, wherein R⁴ is H.

An embodiment of the present invention provides compounds according to formula I as described herein, wherein R⁵ is H.

An embodiment of the present invention provides compounds according to formula I as described herein, wherein R⁷ is alkyl.

An embodiment of the present invention provides compounds according to formula I as described herein, wherein W is a piperidine ring substituted with alkyl.

An embodiment of the present invention provides compounds according to formula I, wherein
A₃ is CR³;
A₄ is CR⁴;
A₅ is CR⁵;
Y is N or CH;
R¹ is alkyl, cyano, or H;
R² is alkyl, cyano, H or halo;
R³ is H;
R⁴ is H or halo;
R⁵ is H or halo;
R⁶ is H;
R⁷ is alkyl or haloalkyl;
W is a piperidine ring substituted with alkyl;
and pharmaceutically acceptable salts thereof.

An embodiment of the present invention provides compounds according to formula I, wherein
A₃ is CR³;
A₄ is CR⁴;
A₅ is CR⁵;
Y is N or CH;
Riis H;
R² is H or halo;
R³ is H;
R⁴ is H;
R⁵ is H;
R⁶ is H;
R⁷ is alkyl or haloalkyl;
W is a piperidine ring substituted with alkyl;
and pharmaceutically acceptable salts thereof.

An embodiment of the present invention provides compounds according to formula I, wherein
A₃ is CR³;
A₄ is CR⁴;
A₅ is CR⁵;
Y is N or CH;
R¹ is H;
R² is H or halo;
R³ is H;
R⁴ is H;
R⁵ is H;
R⁶ is H;
R⁷ is alkyl;
W is a piperidine ring substituted with alkyl;
and pharmaceutically acceptable salts thereof.

Particular examples of compounds of formula I as described herein are selected from
N-[(3R)-1-Ethyl-3-piperidyl]-6-(1H-indol-6-yl)-5-methyl-pyridazin-3-amine;
N-[(3R)-1-Ethyl-3-piperidyl]-6-(3-fluoro-1H-indol-6-yl)-5-methyl-pyridazin-3-amine;
N-[(3R)-1-Ethyl-3-piperidyl]-6-(5-fluoro-1H-indol-6-yl)-5-methyl-pyridazin-3-amine;
N-[(3R)-1-Ethyl-3-piperidyl]-5-methyl-6-(3-methyl-1H-indol-6-yl)pyridazin-3-amine;
6-(3-Chloro-1H-indol-6-yl)-N-[(3R)-1-ethyl-3-piperidyl]-5-methyl-pyridazin-3-amine;
N-[(3R)-1-Ethyl-3-piperidyl]-5-methyl-6-(2-methyl-1H-indol-6-yl)pyridazin-3-amine;
6-[6-[[(3R)-1-Ethyl-3-piperidyl]amino]-4-methyl-pyridazin-3-yl]-1H-indole-3-carbonitrile;
6-[6-[[(3R)-1-Ethyl-3-piperidyl]amino]-4-methyl-pyridazin-3-yl]-1H-indole-2-carbonitrile;
6-(3-Fluoro-1H-indol-6-yl)-5-methyl-N-[(3R)-1-methyl-3-piperidyl]pyridazin-3-amine;
N-[(3R)-1-Ethyl-3-piperidyl]-6-(1H-indol-6-yl)-5-methyl-pyridazin-3-amine;
N-[(3R)-1-Ethyl-3-piperidyl]-6-(3-fluoro-1H-indol-6-yl)-5-(trifluoromethyl)pyridazin-3-amine;
5-Ethyl-N-[(3R)-1-ethyl-3-piperidyl]-6-(3-fluoro-1H-indol-6-yl)pyridazin-3-amine;
N-[(3R)-1-Ethyl-3-piperidyl]-6-(1H-indol-6-yl)-5-methyl-1,2,4-triazin-3-amine;
N-[(3R)-1-Ethyl-3-piperidyl]-6-(3-fluoro-1H-indol-6-yl)-5-methyl-1,2,4-triazin-3-amine;
and pharmaceutically acceptable salts thereof.

Other particular examples of formula I as described herein are selected from
N-[(3R)-1-Ethyl-3-piperidyl]-6-(5-methyl-1H-indol-6-yl)pyridazin-3-amine;
3-[[6-(3-Fluoro-1H-indol-6-yl)-5-methyl-pyridazin-3-yl]amino]-1-methyl-cyclobutanol;
and pharmaceutically acceptable salts thereof.

Preferred examples of compounds of formula I as described herein are selected from
N-[(3R)-1-Ethyl-3-piperidyl]-6-(1H-indol-6-yl)-5-methyl-pyridazin-3-amine;
N-[(3R)-1-Ethyl-3-piperidyl]-6-(3-fluoro-1H-indol-6-yl)-5-methyl-pyridazin-3-amine;
6-(3-Fluoro-1H-indol-6-yl)-5-methyl-N-[(3R)-1-methyl-3-piperidyl]pyridazin-3-amine;
N-[(3R)-1-Ethyl-3-piperidyl]-6-(3-fluoro-1H-indol-6-yl)-5-(trifluoromethyl)pyridazin-3-amine;
N-[(3R)-1-Ethyl-3-piperidyl]-6-(1H-indol-6-yl)-5-methyl-1,2,4-triazin-3-amine;
N-[(3R)-1-Ethyl-3-piperidyl]-6-(3-fluoro-1H-indol-6-yl)-5-methyl-1,2,4-triazin-3-amine;
and pharmaceutically acceptable salts thereof.

Most preferred examples of compounds of formula I as described herein are selected from
N-[(3R)-1-Ethyl-3-piperidyl]-6-(3-fluoro-1H-indol-6-yl)-5-methyl-pyridazin-3-amine;
N-[(3R)-1-Ethyl-3-piperidyl]-6-(1H-indol-6-yl)-5-methyl-1,2,4-triazin-3-amine;
N-[(3R)-1-Ethyl-3-piperidyl]-6-(3-fluoro-1H-indol-6-yl)-5-methyl-1,2,4-triazin-3-amine;
and pharmaceutically acceptable salts thereof.

An embodiment of the present invention provides compounds according to formula Ic, wherein the compound of formula Ic is a compound of formula Ib

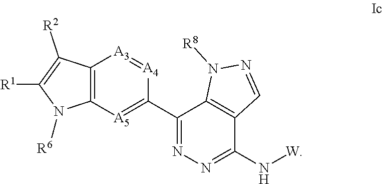

An embodiment of the present invention provides compounds according to formula Ic as described herein, wherein
A₃ is selected from N or CR³;
A₄ is selected from N or CR⁴;
A₅ is selected from N or CR⁵;
R¹ is selected from alkyl, cyano, or H;
R² is selected from alkyl, cyano, H or halo;
R³ is H;
R⁴ is selected from H, alkyl, alkoxy or halo;
R⁵ is selected from H or halo;
R⁶ is H;
R⁸ is H or alkyl;
W is optionally substituted cycloalkyl or optionally substituted heterocycle ring comprising a single N heteroatom, wherein optionally substituted cycloalkyl can be substituted with 1 or 2 substituents independently selected from H, OH or alkyl, and optionally substituted heterocycle ring comprising a single N heteroatom can be substituted with alkyl or haloalkyl;
and pharmaceutically acceptable salts thereof.

An embodiment of the present invention provides compounds according to formula Ic as described herein, wherein R¹ is H.

An embodiment of the present invention provides compounds according to formula Ic as described herein, wherein R² is H.

An embodiment of the present invention provides compounds according to formula Ic as described herein, wherein A₃ is CR³.

An embodiment of the present invention provides compounds according to formula Ic as described herein, wherein A₄ is CR⁴.

An embodiment of the present invention provides compounds according to formula Ic as described herein, wherein A₅ is CR⁵.

An embodiment of the present invention provides compounds according to formula Ic as described herein, wherein R⁴ is H.

An embodiment of the present invention provides compounds according to formula Ic as described herein, wherein R⁵ is H.

An embodiment of the present invention provides compounds according to formula Ic as described herein, wherein R⁸ is alkyl.

An embodiment of the present invention provides compounds according to formula Ic as described herein, wherein W is a piperidine ring substituted with alkyl.

An embodiment of the present invention provides compounds according to formula Ic, wherein
A₃ is CR³;
A₄ is CR⁴;
A₅ is CR⁵;
R¹ is H;
R² is H;
R³ is H;
R⁴ is H;
R⁵ is H;
R⁶ is H;
R⁸ is alkyl or H;
W is a piperidine ring substituted with alkyl;
and pharmaceutically acceptable salts thereof.

An embodiment of the present invention provides compounds according to formula Ic, wherein
A₃ is CR³;
A₄ is CR⁴;
A₅ is CR⁵;
R¹ is H;
R² is H;
R³ is H;
R⁴ is H;
R⁵ is H;
R⁶ is H;
R⁸ is alkyl;
W is a piperidine ring substituted with alkyl;
and pharmaceutically acceptable salts thereof.

A particular example of a compound of formula Ib as described herein is N-[(3R)-1-Ethyl-3-piperidyl]-7-(1H-indol-6-yl)-1-methyl-pyrazolo[3,4-d]pyridazin-4-amine and pharmaceutically acceptable salts thereof.

Particular example of a compound of formula Ic as described herein is N-[(3R)-1-Ethyl-3-piperidyl]-7-(1H-indol-6-yl)-1-methyl-pyrazolo[3,4-d]pyridazin-4-amine and pharmaceutically acceptable salts thereof.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula Ib may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula Ib is formulated in an acetate buffer, at pH 5. In another embodiment, the compound of formula Ib is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula Ib and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula Ib as described herein for use as a therapeutically active substance.

An embodiment of the present invention is a compound according to formula Ib as described herein for use in the treatment or prevention of a disease, disorder or condition, wherein the disease, disorder or condition is responsive to NLRP3 inhibition.

An embodiment of the present invention is a compound according to formula Ib as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3

In another embodiment, the disease, disorder or condition is selected from:
(i) cancer;
(ii) an infection;
(iii) a central nervous system disease;
(iv) a cardiovascular disease;
(v) a liver disease;
(vi) an ocular disease; or
(vii) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia; (vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, Legionella, Lyme disease, influenza A, Epstein-Barr virus infection, viral encephalitis/aseptic meningitis, or pelvic inflammatory disease;
(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xv) a cancer, including those cancers listed above;
(xvi) a burn, wound, trauma, haemorrhage or stroke;
(xvii) radiation exposure;
(xviii) a metabolic disease such as type 2 diabetes (T2D), atherosclerosis, obesity, gout or pseudo-gout; and/or
(xix) pain such as inflammatory hyperalgesia, pelvic pain, allodynia, neuropathic pain, or cancer-induced bone pain.

An embodiment of the present invention is the use of a compound according to formula Ib as described herein in the treatment or prophylaxis of a disease, disorder or condition selected from Alzheimer's disease and Parkinson's disease.

An embodiment of the present invention is the use a compound according to formula Ib as described herein for use in the treatment or prophylaxis of a disease, disorder or condition selected from Asthma or COPD.

An embodiment of the present invention is the use a compound according to formula Ib as described herein for use in the treatment or prophylaxis of a disease, disorder or condition selected from inflammatory bowel disease (including Crohn's disease and ulcerative colitis).

An embodiment of the present invention is a compound according to formula Ib as described herein for the treatment or prophylaxis of a disease, disorder or condition selected from Alzheimer's disease and Parkinson's disease.

An embodiment of the present invention is a compound according to formula Ib as described herein for the treatment or prophylaxis of a disease, disorder or condition selected from Asthma or COPD.

An embodiment of the present invention is a compound according to formula Ib as described herein for the treatment or prophylaxis of a disease, disorder or condition selected from inflammatory bowel disease (including Crohn's disease and ulcerative colitis).

An embodiment of the present invention is the use of a compound according to formula Ib as described herein for preparation of a medicament for the treatment or prophylaxis of a disease, disorder or condition selected from Alzheimer's disease and Parkinson's disease.

An embodiment of the present invention is the use of a compound according to formula Ib as described herein for the preparation of a medicament for the treatment or prophylaxis of a disease, disorder or condition selected from Asthma or COPD.

An embodiment of the present invention is the use of a compound according to formula Ib as described herein for the preparation of a medicament for the treatment or prophylaxis of a disease, disorder or condition selected from inflammatory bowel disease (including Crohn's disease and ulcerative colitis).

An embodiment of the present invention is a method of treatment or prophylaxis of a disease, disorder or condition selected from Alzheimer's disease and Parkinson's disease, which method comprises administering an effective amount of a compound according to formula Ib as described herein.

An embodiment of the present invention is a method of treatment or prophylaxis of a disease, disorder or condition selected from Asthma or COPD, which method comprises administering an effective amount of a compound according to formula Ib as described herein.

An embodiment of the present invention is a method of treatment or prophylaxis of a disease, disorder or condition selected from inflammatory bowel disease (including Crohn's disease and ulcerative colitis), which method comprises administering an effective amount of a compound according to formula Ib as described herein.

An embodiment of the present invention relates to a method of inhibiting NLRP3, which method comprises administering an effective amount of a compound according to formula Ib as described herein.

Also an embodiment of the present invention are compounds of formula Ib as described herein, when manufactured according to any one of the described processes.

An embodiment of the present invention is a pharmaceutical composition comprising a compound according to formula Ib as described herein and a therapeutically inert carrier.

An embodiment of the present invention is a compound according to formula Ib as described herein for the treatment or prophylaxis of a disease, disorder or condition selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hours at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50p of resazurin (Sigma #R7017) (FAC 100 μM resazurin in RPMI medium without FBS) and incubate plates for a further 1-2 hours at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hours at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

TABLE 1
NLRP3 inhibitory activity

| | | THP-1 | Human whole |
|---|---|---|---|
| | | pyroptosis | blood IL-1β |
| Example | | assay IC₅₀ | Assay IC₅₀ |
| No. | Name | (nM) | (nM) |
| 1 | N-[(3R)-1-Ethyl-3-piperidyl]-6-(1H-indol-6-yl)- | 5.1 | 17.1 |
| | 5-methyl-pyridazin-3-amine | | |
| 2 | N-[(3R)-1-Ethyl-3-piperidyl]-6-(3-fluoro-1H- | 2.7 | 9.5 |
| | indol-6-yl)-5-methyl-pyridazin-3-amine | | |
| 3 | N-[(3R)-1-Ethyl-3-piperidyl]-6-(5-fluoro- | 9.2 | 48.6 |
| | 1H-indol-6-yl)-5-methyl-pyridazin-3-amine | | |
| 4 | N-[(3R)-1-Ethyl-3-piperidyl]-5-methyl-6-(3- | 43.7 | 233.5 |
| | methyl-1H-indol-6-yl)pyridazin-3-amine | | |
| 5 | 6-(3-Chloro-1H-indol-6-yl)-N-[(3R)-1-ethyl- | | |
| | 3-piperidyl]-5-methyl-pyridazin-3-amine | | |
| 6 | N-[(3R)-1-Ethyl-3-piperidyl]-5-methyl-6-(2- | 12.3 | 56.2 |
| | methyl-1H-indol-6-yl)pyridazin-3-amine | | |
| 7 | 6-[6-[[(3R)-1-Ethyl-3-piperidyl]amino]-4- | 132.6 | |
| | methyl-pyridazin-3-yl]-1H-indole-3- | | |
| | carbonitrile | | |
| 8 | 6-[6-[[(3R)-1-Ethyl-3-piperidyl]amino]-4- | 122.8 | |
| | methyl-pyridazin-3-yl]-1H-indole-2- | | |
| | carbonitrile | | |
| 9 | 6-(3-Fluoro-1H-indol-6-yl)-5-methyl-N- | 4.9 | 26.5 |
| | [(3R)-1-methyl-3-piperidyl]pyridazin-3- | | |
| | amine | | |
| 10 | N-[(3R)-1-Ethyl-3-piperidyl]-6-(1H-indol-6- | 22.6 | 45.1 |
| | yl)-5-methyl-pyridazin-3-amine | | |
| 11 | N-[(3R)-1-Ethyl-3-piperidyl]-6-(3-fluoro-1H- | 5.1 | 44.1 |
| | indol-6-yl)-5-(trifluoromethyl)pyridazin-3- | | |
| | amine | | |
| 12 | 5-Ethyl-N-[(3R)-1-ethyl-3-piperidyl]-6-(3- | 18.3 | 25.4 |
| | fluoro-1H-indol-6-yl)pyridazin-3-amine | | |
| 13 | N-[(3R)-1-Ethyl-3-piperidyl]-6-(1H-indol-6- | 47.3 | 28.6 |
| | yl)-5-methyl-1,2,4-triazin-3-amine | | |
| 14 | N-[(3R)-1-Ethyl-3-piperidyl]-6-(3-fluoro-1H- | 55.9 | 43.2 |
| | indol-6-yl)-5-methyl-1,2,4-triazin-3-amine | | |
| 15 | N-[(3R)-1-Ethyl-3-piperidyl]-7-(1H-indol-6- | 27 | 52 |
| | yl)-1-methyl-pyrazolo[3,4-d]pyridazin-4- | | |
| | amine | | |
| 16 | N-[(3R)-1-Ethyl-3-piperidyl]-6-(5-methyl- | 21 | 113 |
| | 1H-indol-6-yl)pyridazin-3-amine | | |
| 17 | 3-[6-(3-Fluoro-1H-indol-6-yl)-5-methyl- | 493 | |
| | pyridazin-3-yl]amino]-1-methyl-cyclobutanol | | |
| 18A | (3aS,7aR)-1-[6-(3-Fluoro-1H-indol-6-yl)-5- | 2 | 31 |
| | methyl-pyridazin-3-yl]-6-methyl- | | |
| | 3,3a,4,5,7,7a-hexahydro-2H-pyrrolo[2,3- | | |
| | c]pyridine | | |
| 18B | (3aR,7aS)-1-[6-(3-fluoro-1H-indol-6-yl)-5- | 37 | 179 |
| | methyl-pyridazin-3-yl]-6-methyl- | | |
| | 3,3a,4,5,7,7a-hexahydro-2H-pyrrolo[2,3- | | |
| | c]pyridine | | |

The invention will now be illustrated by the following examples which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

All examples and intermediates were prepared under nitrogen atmosphere if not specified otherwise.

### Abbreviations

| | |
|---|---|
| aq. | aqueous |
| DIPEA | N,N-diisopropylethylamine |
| eq | equivalent(s) |
| EtOAc | Ethyl acetate |
| h | hour(s) |
| HPLC | High-performance liquid chromatography |
| LCMS | Liquid chromatography mass spectrometry |
| MeCN | Acetonitrile |
| MeOH | Methanol |
| PE | Petroleum ether |
| Prep. | preparative |
| rt | Room temperature |

### Example 1: N-[(3R)-1-Ethyl-3-piperidyl]-6-(1H-indol-6-yl)-5-methyl-pyridazin-3-amine

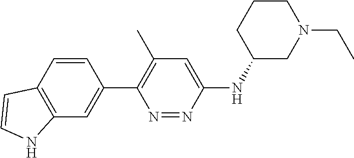 [SMILES: CCN1CCC[C@@H](Nc2cc(C)c(-c3ccc4cc[nH]c4c3)nn2)C1]

### Step A: tert-Butyl (3R)-3-[(6-chloro-5-methyl-pyridazin-3-yl)amino]piperidine-1-carboxylate

To a suspension of 3,6-dichloro-4-methyl-pyridazine (CAS #19064-64-3, 1.60 g, 9.82 mmol, 1.00 eq) and tert-butyl (3R)-3-aminopiperidine-1-carboxylate (CAS #188111-79-7, 3.93 g, 19.6 mmol, 2.00 eq) was added DIPEA (3.26 g, 4.4 mL, 25.2 mmol, 2.57 eq). The reaction mixture was stirred at 120° C. for 72 hours. The reaction mixture was cooled to room temperature and then extracted with ethyl acetate and water. The organic layer was washed with water and brine. The aqueous layers were backextracted with dichloromethane. The combined organic layers were dried over sodium sulfate, filtered and concentrated in vacuo. The crude product was adsorbed on ISOLUTE HM-N and purified by flash chromatography (silica gel, 120 g, gradient 0% to 5% methanol in dichloromethane) to afford the title compound (1.77 g, 52% yield) as a light yellow oil. LCMS: m/z 327.2 [M+H]⁺, ESI pos.

### Step B: 6-Chloro-5-methyl-N-[(3R)-3-piperidyl]pyridazin-3-amine hydrochloride

To a solution of tert-butyl (3R)-3-[(6-chloro-5-methyl-pyridazin-3-yl)amino]piperidine-1-carboxylate (Example 1, step A) (1.65 g, 4.79 mmol, 1.00 eq) in dichloromethane (12 mL) and methanol (6.0 mL) was added dropwise 4 M HCl in dioxane (12 mL, 48.0 mmol, 10.0 eq). The reaction mixture was stirred at room temperature for 16 hours. The reaction mixture was concentrated in vacuo to afford the title compound (1.33 g, 95% yield, 90% purity) as an off-white solid, which was used without further purification. LCMS: m/z 227.2 [M+H]⁺, ESI pos.

### Step C: 6-Chloro-N-[(3R)-1-ethyl-3-piperidyl]-5-methyl-pyridazin-3-amine

To a suspension of 6-chloro-5-methyl-N-[(3R)-3-piperidyl]pyridazin-3-amine hydrochloride (Example 1, step B) (1.32 g, 4.51 mmol, 1.00 eq, 90% purity) in dichloromethane (20 mL) was added acetaldehyde (405.7 mg, 0.460 mL, 9.21 mmol, 2.04 eq) followed by sodium acetate (741 mg, 9.03 mmol, 2.00 eq) under ice-bath cooling. Sodium triacetoxyborohydride (1.44 g, 6.77 mmol, 1.50 eq) was added in three portions at 0° C. The reaction mixture was stirred at 0° C. for 30 minutes and at room temperature for 2.5 hours. The reaction mixture was quenched with saturated aq. NaHCO₃-solution and extracted three times with dichloromethane. The combined organic layers were dried over sodium sulfate, filtered and concentrated in vacuo. The crude product was adsorbed on ISOLUTE HM-N and purified by flash chromatography (silica gel, 40 g, gradient 0% to 10% methanol in dichloromethane) to afford the title compound (901 mg, 74% yield) as a yellow oil. LCMS: m/z 255.2 [M+H]⁺, ESI pos.

### Step D: N-[(3R)-1-Ethyl-3-piperidyl]-6-(1H-indol-6-yl)-5-methyl-pyridazin-3-amine

A mixture of 6-chloro-N-[(3R)-1-ethyl-3-piperidyl]-5-methyl-pyridazin-3-amine (Example 1, step C) (80 mg, 0.30 mmol, 1.00 eq), 6-(4,4,5,5-tetramethyl-1,3,2-dioxaborolan-2-yl)-1H-indole (CAS #642494-36-8, 130 mg, 0.51 mmol, 1.70 eq), potassium carbonate (195 mg, 1.41 mmol, 4.73 eq) and 1,1′-bis(diphenylphosphino)ferrocene-palladium(ii) dichloride dichloromethane complex (28 mg, 0.03 mmol, 0.11 eq) in 1,4-dioxane (2.0 mL) and water (1.0 mL) was flushed with argon and stirred at 90° C. for 16 hours. The reaction mixture was cooled to room temperature and extracted with ethyl acetate and water. The aqueous layer was back extracted with ethyl acetate. The organic layers were washed twice with water and once with brine. The combined organic layers were dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 9:1:0.05) in dichloromethane). The residue was adsorbed on ISOLUTE HM-N and purified again by flash chromatography (silica gel, 12 g, gradient 0% to 20% methanol in dichloromethane). All fractions containing product were combined and concentrated in vacuo to afford the title compound (61 mg, 58% yield) as a light brown foam. LCMS: m/z 336.3 [M+H]⁺, ESI pos.

### Example 2: N-[(3R)-1-Ethyl-3-piperidyl]-6-(3-fluoro-1H-indol-6-yl)-5-methyl-pyridazin-3-amine

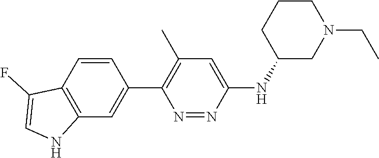 [SMILES: CCN1CCC[C@@H](Nc2cc(C)c(-c3ccc4c(F)c[nH]c4c3)nn2)C1]

### Step A: 3-Fluoro-6-(4,4,5,5-tetramethyl-1,3,2-dioxaborolan-2-yl)-1H-indole

To a solution of commercially available 6-bromo-3-fluoro-1H-indole (CAS #2568045-20-3, 296 mg, 1.31 mmol, 1.00 eq) and bis(pinacolato)diboron (400 mg, 1.58 mmol, 1.20 eq) in 1,4-dioxane (3.1 mL) were added potassium acetate (387 mg, 3.94 mmol, 3.00 eq) followed by 1,1′-bis(diphenylphosphino)ferrocene-palladium(II)dichloride dichloromethane complex (109 mg, 0.13 mmol, 0.10 eq). The reaction mixture was flushed with argon and stirred at 90° C. for 16 hours. The reaction mixture was then cooled to room temperature, adsorbed on ISOLUTE HM-N and purified by flash chromatography (silica gel, 25 g, 0% to 10% ethyl acetate in heptane) to afford the title compound (224 mg, 62% yield) as a dark brown solid. LCMS: m/z 262.1 [M+H]⁺, ESI pos.

### Step B: N-[(3R)-1-Ethyl-3-piperidyl]-6-(3-fluoro-1H-indol-6-yl)-5-methyl-pyridazin-3-amine

A mixture of aforementioned 6-chloro-N-[(3R)-1-ethyl-3-piperidyl]-5-methyl-pyridazin-3-amine (Example 1, step C) (75 mg, 0.28 mmol, 1.00 eq), 3-fluoro-6-(4,4,5,5-tetramethyl-1,3,2-dioxaborolan-2-yl)-1H-indole (Example 2, step A) (124 mg, 0.45 mmol, 1.61 eq), potassium carbonate (180 mg, 1.30 mmol, 4.66 eq) and 1,1′-bis(diphenylphosphino)ferrocene-palladium(ii) dichloride dichloromethane complex (28 mg, 0.03 mmol, 0.12 eq) in 1,4-dioxane (1.8 mL) and water (0.90 mL) was flushed with argon and stirred at 90° C. for 16 hours. The reaction mixture was cooled to room temperature and extracted with ethyl acetate and water. The aqueous layer was back extracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 9:1:0.05) in dichloromethane). All fractions containing product were combined and concentrated in vacuo to afford the title compound (77 mg, 74% yield) as a brown foam. LCMS: m/z 354.2 [M+H]⁺, ESI pos.

### Example 3: N-[(3R)-1-ethyl-3-piperidyl]-6-(5-fluoro-1H-indol-6-yl)-5-methyl-pyridazin-3-amine

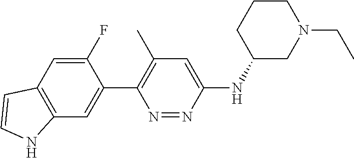 [SMILES: CCN1CCC[C@@H](Nc2cc(C)c(-c3cc4[nH]ccc4cc3F)nn2)C1]

### Step A: 5-Fluoro-6-(4,4,5,5-tetramethyl-1,3,2-dioxaborolan-2-yl)-1H-indole

6-Bromo-5-fluoro-1H-indole (500 mg, 2.34 mmol, 1.0 eq), potassium 2-ethylhexanoate (937 mg, 5.14 mmol, 2.2 eq), palladium acetate (10.7 mg, 0.05 mmol, 0.02 eq) and Xphos (44.5 mg, 0.09 mmol, 0.04 eq) were dissolved in isopropyl acetate (7.5 mL). The reaction mixture was degassed with argon and stirred at 80° C. for 2 h. After cooling to rt, the mixture was filtered using a syringe filter and the solvent was evaporated in vacuo. The title compound was used without further purification in the next step. LCMS: m/z=261.9 [M+H]⁺, ESI pos.

### Step B: N-[(3R)-1-ethyl-3-piperidyl]-6-(5-fluoro-1H-indol-6-yl)-5-methyl-pyridazin-3-amine

A mixture of aforementioned 5-fluoro-6-(4,4,5,5-tetramethyl-1,3,2-dioxaborolan-2-yl)-1H-indole (Example 3, step A) (53.0 mg, 0.30 mmol, 1.5 eq), aforementioned 6-chloro-N-[(3R)-1-ethyl-3-piperidyl]-5-methyl-pyridazin-3-amine (Example 1, step C) (50.0 mg, 0.20 mmol, 1.0 eq), [1,1′-bis(diphenylphosphino)ferrocene]dichloropalladium(ii) (14.3 mg, 0.02 mmol, 0.10 eq) and cesium carbonate (191 mg, 0.59 mmol, 3.0 eq) in 1,4-dioxane (2.1 mL) and water (1.0 mL) was purged with argon and stirred at 90° C. After 5 h, the reaction mixture was cooled to rt and extracted with ethyl acetate and washed with water and brine. The combined organic phases were dried over sodium sulfate, filtered and the solvent was removed under reduced pressure. The crude was purified by preparative HPLC (column: YMC-Triart Cig, 12 nm, 5 μm, 100×30 mm, MeCN/Water+0.1% TEA, with a gradient from 20-80% MeCN) to yield the title compound (18 mg, 23% yield). LC-MS: m/z=354.2 [M+H]⁺, ESI pos.

### Example 4: N-[(3R)-1-Ethyl-3-piperidyl]-5-methyl-6-(3-methyl-1H-indol-6-yl)pyridazin-3-amine

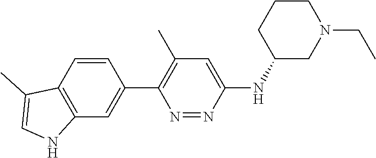 [SMILES: CCN1CCC[C@@H](Nc2cc(C)c(-c3ccc4c(C)c[nH]c4c3)nn2)C1]

To a 2 mL microwave vial was added aforementioned 6-chloro-N-[(3R)-1-ethyl-3-piperidyl]-5-methyl-pyridazin-3-amine (Example 1, step C) (50 mg, 0.196 mmol, 1.00 eq), commercially available 3-methyl-6-(4,4,5,5-tetramethyl-1,3,2-dioxaborolan-2-yl)-1H-indole (CAS #1300582-52-8, 60.6 mg, 0.236 mmol, 1.200 eq), 1,1′-bis(diphenylphosphino)ferrocene-palladium(ii) dichloride dichloromethane complex (8.01 mg, 0.010 mmol, 0.050 eq) and potassium carbonate (2.25 M aq. solution) (260 μL, 0.585 mmol, 2.98 eq) in methanol (1.7 mL). The reaction mixture was sparged with argon while being sonicated for 5 min. The vial was capped and heated in the microwave at 80° C. for 2 h. The reaction progress was monitored by LCMS and showed 54% conversion. The reaction mixture was diluted with MeOH and filtered using a syringe filter to yield a red solution, which was purified by prep. HPLC (Gemini NX, ACN/Water (Et₃N), basic mode) to yield the title compound (21.7 mg, 31% yield) as white lyophilizate powder. LCMS: m/z=350.3 [M+H]⁺ ESI pos.

### Example 5: 6-(3-Chloro-1H-indol-6-yl)-N-[(3R)-1-ethyl-3-piperidyl]-5-methyl-pyridazin-3-amine

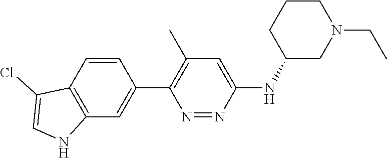 [SMILES: CCN1CCC[C@@H](Nc2cc(C)c(-c3ccc4c(Cl)c[nH]c4c3)nn2)C1]

To a 2 mL microwave vial was added 6-chloro-N-[(3R)-1-ethyl-3-piperidyl]-5-methyl-pyridazin-3-amine (Example 1, step C) (50 mg, 0.196 mmol, 1.000 eq), commercially available 3-chloro-6-(4,4,5,5-tetramethyl-1,3,2-dioxaborolan-2-yl)-1H-indole (CAS #2304635-36-5, 70.8 mg, 0.255 mmol, 1.30 eq), 1,1′-bis(diphenylphosphino)ferrocene-palladium(ii) dichloride dichloromethane complex (16.0 mg, 0.020 mmol, 0.10 eq) and potassium carbonate (2.25 M aq. solution) (436.1 μL, 0.981 mmol, 5.0 eq) in 1,4-dioxane (1.78 mL). The reaction mixture was sparged with argon while being sonicated for 5 min. The vial was capped and heated in the microwave at 90° C. for 2 h. The reaction progress was monitored by LCMS and showed reaction was complete. The reaction mixture was filtered using a syringe filter and solvent was evaporated in a centrifuge. The crude was purified by prep. HPLC (Gemini NX, ACN/Water (Et3N), basic mode) to yield the title compound (13.1 mg, 18% yield) as white lyophilizate powder. LCMS: m/z=368.3 [M−H]⁻, ESI neg.

### Example 6: N-[(3R)-1-Ethyl-3-piperidyl]-5-methyl-6-(2-methyl-1H-indol-6-yl)pyridazin-3-amine

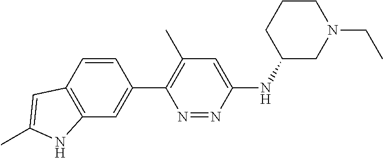 [SMILES: CCN1CCC[C@@H](Nc2cc(C)c(-c3ccc4cc(C)[nH]c4c3)nn2)C1]

To a 5 mL microwave vial was added 6-chloro-N-[(3R)-1-ethyl-3-piperidyl]-5-methyl-pyridazin-3-amine (Example 1, step C) (50 mg, 0.196 mmol, 1.000 eq), commercially available 2-methyl-6-(4,4,5,5-tetramethyl-1,3,2-dioxaborolan-2-yl)-1H-indole (CAS #1650548-44-9, 60.56 mg, 0.236 mmol, 1.20 eq), 1,1′-bis(diphenylphosphino)ferrocene-palladium(ii) dichloride dichloromethane complex (16.0 mg, 0.020 mmol, 0.100 eq) and potassium carbonate (2.25 M aq. solution) (261.7 μL, 0.589 mmol, 3.000 eq) in methanol (3 mL). The reaction mixture was sparged with argon while being sonicated for 3 min. The vial was capped and heated in the microwave at 80° C. for 4 h. The reaction progress was monitored by LCMS and showed 71% conversion. The reaction mixture was filtered using a syringe filter and solvent was evaporated in a centrifuge. The crude was purified by prep. HPLC (YMC Triart, ACN/Water (HCOOH), acidic mode) to yield the title compound (7.0 mg, 10% yield) as light brown solid. LCMS: m/z=350.2 [M+H], ESI pos.

### Example 7: 6-[6-[[(3R)-1-Ethyl-3-piperidyl]amino]-4-methyl-pyridazin-3-yl]-1H-indole-3-carbonitrile

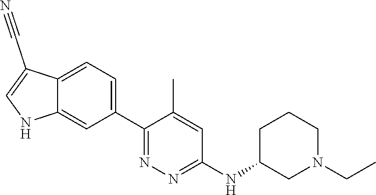 [SMILES: CCN1CCC[C@@H](Nc2cc(C)c(-c3ccc4c(C#N)c[nH]c4c3)nn2)C1]

### Step A: (3R)-3-[(6-Bromo-5-methyl-pyridazin-3-yl)amino]piperidine-1-carboxylic acid tert-butyl ester

In a sealed tube, a mixture of commercially available 3-bromo-6-chloro-4-methyl-pyridazine (CAS #89283-90-9, 3.0 g, 14.5 mmol, 1.0 eq), DIPEA (4.67 g, 6.31 mL, 36.2 mmol, 2.5 eq) and (3R)-3-aminopiperidine-1-carboxylic acid tert-butyl ester (CAS #188111-79-7, 5.79 g, 5.57 mL, 28.9 mmol, 20. eq) was stirred at 120° C. for 18 h. The reaction mixture was cooled to room temperature and extracted with EtOAc and water. The aqueous layer was back extracted with EtOAc. The organic layers were washed with water and brine. The combined organic layers were dried over sodium sulfate, filtered and concentrated in vacuo. The residue was adsorbed on ISOLUTE HM-N and purified by flash chromatography (silica gel, 140 g, gradient: 0% to 2% methanol in dichloromethane). All fractions containing product were combined and concentrated in vacuo to afford the title compound (988 mg, 18%) as white powder. LCMS: m/z: 371.2 ([{79Br}M+H]+), 373.2 ([{81Br}M+H]⁺), ESI pos.

### Step B: (6-Bromo-5-methyl-pyridazin-3-yl)-[(3R)-3-piperidyl]amine·1:1 Hydrogen Chloride

To a solution of (3R)-3-[(6-bromo-5-methyl-pyridazin-3-yl)amino]piperidine-1-carboxylic acid tert-butyl ester (Example 7, step A) (988 mg, 2.66 mmol, 1.0 eq) in dichloromethane (18 mL) and methanol (9 mL) was added at room temperature dropwise 4 M hydrochloric acid in 1,4-dioxane (6.65 mL, 26.6 mmol, 10.0 eq). The reaction mixture was stirred at 23° C. for 16 h. The reaction mixture was then concentrated in vacuo to give the crude title compound (886 mg, 97%) as light yellow powder. LCMS: m/z: 271.1 ([{79Br}M+H]⁺), 273.1 ([{81Br}M+H]⁺), ESI pos.

### Step C: (6-Bromo-5-methyl-pyridazin-3-yl)-[(3R)-1-ethyl-3-piperidyl]amine

To a suspension of (6-bromo-5-methyl-pyridazin-3-yl)-[(3R)-3-piperidyl]amine 0.1:1 hydrogen chloride (Example 7, step B) (886 mg, 2.88 mmol, 1.0 eq) in dichloromethane, extra dry (13 mL) was added acetaldehyde (317.2 mg, 404.07 μL, 7.2 mmol, 2.5 eq) followed by sodium acetate (590.7 mg, 7.20 mmol, 2.5 eq) under ice-bath cooling. Sodium triacetoxyborohydride (1.10 g, 5.18 mmol, 1.8 eq) was added at 0° C. The reaction mixture was stirred at 0° C. for 15 min and at room temperature for 2 h. The reaction mixture was carefully basified with NaHCO₃-solution (50 mL) and then extracted with dichloromethane (3×80 mL). The combined organic layers were dried over sodium sulfate, filtered and concentrated in vacuo the afford the crude title compound (667 mg, 74%) as orange viscous oil. LCMS: m/z 299.1 ([{³⁵Cl}M+H]+), 301.1 ([{³⁷Cl}M+H]+), ESI pos.

### Preparation of Boronic Ester: 6-(4,4,5,5-tetramethyl-1,3,2-dioxaborolan-2-yl)-1H-indole-3-carbonitrile

To a solution of commercially available 6-bromo-1H-indole-3-carbonitrile (CAS #224434-83-7, 500 mg, 2.26 mmol, 1.0 eq) and bis(pinacolato) diboron (689.3 mg, 2.71 mmol, 1.2 eq) in 1,4-dioxane (5.25 mL) was added potassium acetate (666 mg, 548.13 μL, 6.79 mmol, 3.0 eq) followed by [1,1′-bis(diphenylphosphino)ferrocene]dichloropalladium(ii) (165.51 mg, 0.226 mmol, 0.1 eq). The reaction mixture was flushed with argon and stirred at 90° C. overnight. The reaction mixture was cooled to room temperature, adsorbed on ISOLUTE HM-N and purified by flash chromatography (silica gel, 25 g, 0% to 50% EtOAc in heptane) to afford the title compound (372 mg, 61%) as white powder. LCMS: m/z 269.1 [M+H]+, ESI pos.

### Step D: 6-[6-[[(3R)-1-ethyl-3-piperidyl]amino]-4-methyl-pyridazin-3-yl]-1H-indole-3-carbonitrile

A mixture of (6-bromo-5-methyl-pyridazin-3-yl)-[(3R)-1-ethyl-3-piperidyl]amine (80.0 mg, 0.267 mmol, 1.0 eq), 6-(4,4,5,5-tetramethyl-1,3,2-dioxaborolan-2-yl)-1H-indole-3-carbonitrile (Example 7, see above) (100.4 mg, 0.374 mmol, 1.4 eq), potassium carbonate (166.3 mg, 1.2 mmol, 4.5 eq) and [1,1′-bis(diphenylphosphino)ferrocene]dichloro-palladium (ii)dichloromethane adduct (32.8 mg, 0.040 mmol, 0.150 eq) in 1,4-dioxane (1 mL) and water (0.5 mL) was flushed with argon and stirred at 95° C. overnight. The residue was adsorbed on ISOLUTE HM-N and purified by flash chromatography (silica gel, 12 g, gradient 0% to 20% (dichloromethane:methanol:NH₄OH 9:1:0.05) in dichloromethane). All fractions containing product were combined and concentrated in vacuo. The residue was triturated with EtOAc/heptane to afford the title compound (69 mg, 68.01%) as pink powder as light pink powder. LCMS: m/z: [M−H]⁺=359.3, ESI neg.

### Example 8: 6-[6-[[(3R)-1-Ethyl-3-piperidyl]amino]-4-methyl-pyridazin-3-yl]-1H-indole-2-carbonitrile

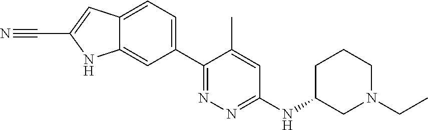 [SMILES: CCN1CCC[C@@H](Nc2cc(C)c(-c3ccc4cc(C#N)[nH]c4c3)nn2)C1]

### Step A: 6-(4,4,5,5-Tetramethyl-1,3,2-dioxaborolan-2-yl)-1H-indole-2-carbonitrile

To a solution of 6-bromo-1H-indole-2-carbonitrile (CAS #1420537-60-5, 500 mg, 2.26 mmol, 1.0 eq) and bis(pinacolato)diboron (688.3 mg, 2.71 mmol, 1.2 eq) in 1,4-dioxane (5.25 mL) were added potassium acetate (665.2 mg, 6.78 mmol, 3.0 eq) followed by 1,1′-bis(diphenylphosphino)ferrocene-palladium(II)dichloride dichloromethane complex (187.2 mg, 0.226 mmol, 0.1 eq). The reaction mixture was flushed with argon and stirred at 90° C. overnight. The reaction mixture was then cooled to room temperature (no full conversion), adsorbed on ISOLUTE HM-N and purified by flash chromatography (silica gel, 25 g, 0% to 50% ethyl acetate in heptane) to give the title compound (85 mg, 14%) as a light yellow crystalline. LCMS: m/z 267.1 [M−H]+, ESI neg.

### Step B: 6-[6-[[(3R)-1-Ethyl-3-piperidyl]amino]-4-methyl-pyridazin-3-yl]-1H-indole-2-carbonitrile

A mixture of aforementioned (6-bromo-5-methyl-pyridazin-3-yl)-[(3R)-1-ethyl-3-piperidyl]amine (80.0 mg, 0.267 mmol, 1.0 eq), 6-(4,4,5,5-tetramethyl-1,3,2-dioxaborolan-2-yl)-1H-indole-2-carbonitrile (Example 10, step A) (86.0 mg, 0.321 mmol, 1.2 eq), potassium carbonate (166.3 mg, 1.20 mmol, 4.50 eq) and [1,1′-bis(diphenylphosphino)ferrocene]dichloro-palladium (ii)dichloromethane adduct (32.8 mg, 0.040 mmol, 0.15 eq) in 1,4-dioxane (1 mL) and water (0.5 mL) was flushed with argon and stirred at 95° C. overnight. The residue was adsorbed on ISOLUTE HM-N and purified by flash chromatography (silica gel, 12 g, gradient (dichloromethane:methanol:NH₄OH 9:1:0.05) in dichloromethane). All fractions containing product were combined and concentrated in vacuo. The residue was triturated with EtOAc/heptane to afford the title compound (49 mg, 48%) as light brown powder. LCMS: m/z: [M−H]⁺=359.3, ESI neg.

### Example 9: 6-(3-Fluoro-1H-indol-6-yl)-5-methyl-N-[(3R)-1-methyl-3-piperidyl]pyridazin-3-amine

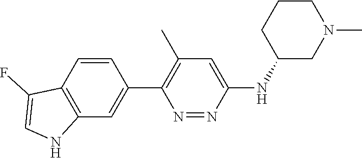 [SMILES: Cc1cc(N[C@@H]2CCCN(C)C2)nnc1-c1ccc2c(F)c[nH]c2c1]

A mixture of 6-chloro-5-methyl-N-[(3R)-1-methyl-3-piperidyl]pyridazin-3-amine (CAS #2557359-57-4, 50 mg, 0.20 mmol, 1.00 eq), 3-fluoro-6-(4,4,5,5-tetramethyl-1,3,2-dioxaborolan-2-yl)-1H-indole (Example 2, step A) (82 mg, 0.30 mmol, 1.51 eq), potassium carbonate (130 mg, 0.94 mmol, 4.77 eq) and 1,1′-bis(diphenylphosphino)ferrocene-palladium(ii) dichloride dichloromethane complex (20 mg, 0.02 mmol, 0.12 eq) in 1,4-dioxane (1.3 mL) and water (0.65 mL) was flushed with argon and stirred at 90° C. for 16 hours. The reaction mixture was cooled to room temperature and extracted with ethyl acetate and water. The aqueous layer was back extracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, gradient 0% to 100% (dichloromethane:methanol:NH₄OH 9:1:0.05) in dichloromethane). All fractions containing product were combined and concentrated in vacuo. The residue was adsorbed on ISOLUTE HM-N and purified again by flash chromatography (Si-amine, 12 g, gradient 0% to 5% methanol in ethyl acetate) to afford the title compound (40 mg, 57% yield) as a brown foam. LCMS: m/z 340.2 [M+H]⁺, ESI pos.

### Example 10: N-[(3R)-1-ethyl-3-piperidyl]-6-(7-fluoro-1H-indol-6-yl)-5-methyl-pyridazin-3-amine

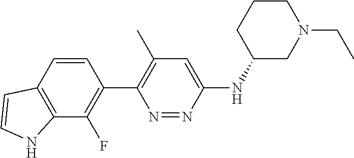 [SMILES: CCN1CCC[C@@H](Nc2cc(C)c(-c3ccc4cc[nH]c4c3F)nn2)C1]

Step A: 7-Fluoro-6-(4,4,5,5-tetramethyl-1,3,2-dioxaborolan-2-yl)-1H-indole Commercially available 6-bromo-5-fluoro-1H-indole (CAS #936901-94-9, 500 mg, 2.34 mmol, 1.0 eq), commercially available potassium 2-ethylhexanoate (CAS #3164-85-0, 936.9 mg, 5.14 mmol, 2.2 eq), palladium acetate (10.7 mg, 0.05 mmol, 0.020 eq) and Xphos (44.5 mg, 0.093 mmol, 0.04 eq) were dissolved in isopropyl acetate (7.5 mL). The mixture was first purged with argon and then stirred at 80° C. for 1 h. Afterwards, the reaction mixture was filtered using a syringe filter and solvent was evaporated in vacuo. The crude product was used without further purification in the next step. LCMS: m/z=262.0 [M+H]⁺, ESI pos.

### Step B: N-[(3R)-1-Ethyl-3-piperidyl]-6-(1H-indol-6-yl)-5-methyl-pyridazin-3-amine

A mixture of aforementioned 7-fluoro-6-(4,4,5,5-tetramethyl-1,3,2-dioxaborolan-2-yl)-1H-indole (Example 11, step A) (97.6 mg, 0.54 mmol, 1.0 eq), 6-chloro-N-[(3R)-1-ethyl-3-piperidyl]-5-methyl-pyridazin-3-amine (Example 1, step C) (97 mg, 0.38 mmol, 0.7 eq), [1,1′-bis(diphenylphosphino)ferrocene]dichloropalladium(ii) (20 mg, 0.03 mmol, 0.05 eq) and cesium carbonate (533 mg, 1.6 mmol, 3.0 eq) in 1,4-dioxane (5.8 mL) and water (2.9 mL) was flushed with argon and stirred at 90° C. After cooling to rt the mixture was passed over a syringe filter and the solvent was removed under reduced pressure. Then, the same amount of reagents and solvents as stated above were added again and the reaction was heated at 90° C. for 5 d. Then, the reaction mixture was extracted with ethyl acetate and washed twice with water. The aqueous layer was back extracted with EtOAc. The crude was purified by preparative HPLC (column: YMC-Triart C₁₈, 12 nm, 5 μm, 100×30 mm, MeCN/(Water+0.1% trimethylamine), gradient 20% to 50% MeCN) to afford the title compound (34.3 mg, 16% yield). LCMS: m/z=354.3 [M+H]⁺.

### Example 11: N-[(3R)-1-Ethyl-3-piperidyl]-6-(3-fluoro-1H-indol-6-yl)-5-(trifluoromethyl)pyridazin-3-amine

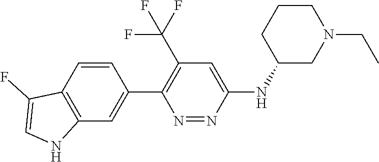 [SMILES: CCN1CCC[C@@H](Nc2cc(C(F)(F)F)c(-c3ccc4c(F)c[nH]c4c3)nn2)C1]

### Step A: tert-Butyl (3R)-3-[[6-chloro-5-(trifluoromethyl)pyridazin-3-yl]amino]piperidine-1-carboxylate and tert-butyl (3R)-3-[[6-chloro-4-(trifluoromethyl)pyridazin-3-yl]amino]piperidine-1-carboxylate

A neat mixture of commercially available 3,6-dichloro-4-(trifluoromethyl)pyridazine (CAS #1057672-68-0, 1.72 g, 7.93 mmol, 1.00 eq) and commercially available (3R)-3-aminopiperidine-1-carboxylic acid tert-butyl ester (CAS #188111-79-7, 3.18 g, 15.9 mmol, 2.00 eq) in N,N-diisopropylethylamine (2.56 g, 3.46 mL, 19.8 mmol, 2.50 eq) was stirred at 130° C. in a sealed tube for 24 h. The warm mixture (ca. 50° C.) was poured into a 500 mL-Erlenmeyer, ethyl acetate (100 mL) was used to transfer the oily mixture, and water (100 mL) was added. Stirred at rt for 20 min to dissolve all ingredients. Then, the mixture was extracted with ethyl acetate and washed twice with water. The aqueous layer was back extracted with EtOAc and finaly brine. The residue was purified by flash chromatography (SiO₂, 0-50% ethyl acetate in heptane) to give the two title compounds: tert-Butyl (3R)-3-[[6-chloro-5-(trifluoromethyl)pyridazin-3-yl]amino]piperidine-1-carboxylate as a light yellow foam (1.67 g. 55%) and tert-butyl (3R)-3-[[6-chloro-4-(trifluoromethyl)pyridazin-3-yl]amino]piperidine-1-carboxylate as a light yellow oil (1.07 g, 35%). LCMS: m/z=381.1 ([{35Cl}M+H]⁺), 383.1 ([{37Cl}M+H]⁺), ESI pos.

### Step B: 6-Chloro-N-[(3R)-3-piperidyl]-5-(trifluoromethyl)pyridazin-3-amine;hydrochloride

To a solution of aforementioned tert-Butyl (3R)-3-[[6-chloro-5-(trifluoromethyl)pyridazin-3-yl]amino]piperidine-1-carboxylate (Example 11, step A, fraction 1) (1.67 g, 4.39 mmol, 1.00 eq) in dichloromethane (20 mL) and methanol (10 mL) was added via syringe 4 M HCl (in dioxane) (13.2 g, 10.96 mL, 43.9 mmol, 10.0 eq). The clear, yellow reaction solution was stirred at 23° C. for 16 hours. The reaction mixture was concentrated in vacuo to afford the title compound as a 1:1 hydrogen chloride (1.51 g, 98%) as light yellow foam. LCMS: m/z=281.1 ([{35Cl}M+H]+), 283.1 ([{37Cl}M+H]+), ESI pos.

### Step C: 6-Chloro-N-[(3R)-1-ethyl-3-piperidyl]-5-(trifluoromethyl)pyridazin-3-amine

To a suspension of aforementioned 6-chloro-N-[(3R)-3-piperidyl]-5-(trifluoromethyl)pyridazin-3-amine;hydrochloride (Example 11, step B) (1.51 g, 4.29 mmol, 1.00 eq) in dichloromethane, extra dry (30 mL) was added acetaldehyde (471.9 mg, 597.38 μL, 10.7 mmol, 2.50 eq) followed by sodium acetate (878.8 mg, 10.7 mmol, 2.50 eq) under ice-bath cooling. Then sodium triacetoxyborohydride (1.63 g, 7.71 mmol, 1.800 eq) was added at 0° C. The reaction mixture was stirred at 0° C. for 15 min and at room temperature for 2 h (light yellow suspension). LCMS showed complete conversion to product. The reaction mixture was carefully basified with NaHCO₃-solution (50 mL) and then extracted with dichloromethane (3×80 mL). The combined organic layers were dried over sodium sulfate, filtered and concentrated in vacuo. The crude product (1.4 g) was adsorbed on ISOLUTE HM-N and purified by flash chromatography (SiO₂, gradient 0% to 100% (dichloromethane:methanol:NH₄OH 110:10:1) in dichloromethane) to afford the title compound (1.03 g, 76%) as light brown oil. LCMS: m/z=309.1 ([{35Cl}M+H]+), 311.0 ([{37Cl}M+H]⁺), ESI pos.

### Step D: N-[(3R)-1-Ethyl-3-piperidyl]-6-(3-fluoro-1H-indol-6-yl)-5-(trifluoromethyl)pyridazin-3-amine

A mixture of aforementioned 6-chloro-N-[(3R)-1-ethyl-3-piperidyl]-5-(trifluoromethyl)pyridazin-3-amine (Example 11, step C) (40 mg, 129.6 μmol, 1.00 eq), 3-fluoro-6-(4,4,5,5-tetramethyl-1,3,2-dioxaborolan-2-yl)-1H-indole (Example 2, step A) (50.7 mg, 194.3 μmol, 1.50 eq), potassium carbonate (85.95 mg, 621.9 μmol, 4.800 eq) and 1,1′-bis(diphenylphosphino)ferrocene-palladium(ii) dichloride dichloromethane complex (15.9 mg, 19.4 μmol, 0.15 eq) in 1,4-dioxane (1 mL) and water (500 μL) was flushed with argon and stirred at 90° C. overnight. LCMS showed complete conversion to product. The reaction mixture was cooled to room temperature and extracted with ˜30 mL ethyl acetate and ˜15 mL water. The aqueous layer was backextracted with ˜30 mL ethyl acetate. The organic layers were washed with ˜15 mL water and ˜15 mL brine. The combined organic layers were dried over sodium sulfate, filtered and concentrated in vacuo. The crude product was purified by RP HPLC (Gemini NX, 12 nm, 5 μm, 100×30 mm, CAN/water+0.1% TEA) to afford the title product (24 mg, 43%) as light grey amorph freeze-dried solid. LCMS: m/z=408.2 [M+H]+, ESI pos.

### Example 12: 5-Ethyl-N-[(3R)-1-ethyl-3-piperidyl]-6-(3-fluoro-1H-indol-6-yl)pyridazin-3-amine

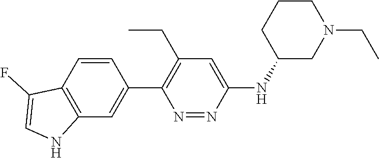 [SMILES: CCc1cc(N[C@@H]2CCCN(CC)C2)nnc1-c1ccc2c(F)c[nH]c2c1]

### Step A: (3R)-3-[(6-Chloro-5-ethyl-pyridazin-3-yl)amino]piperidine-1-carboxylic acid tert-butyl ester and (3R)-3-[(6-chloro-4-ethyl-pyridazin-3-yl)amino]piperidine-1-carboxylic acid tert-butyl ester

In a sealed tube, a neat mixture of (3R)-3-aminopiperidine-1-carboxylic acid tert-butyl ester (CAS #188111-79-7, 4.53 g, 22.6 mmol, 2.00 eq) and 3,6-dichloro-4-ethyl-pyridazine (2.0 g, 11.3 mmol, 1.00 eq) in DIPEA (3.65 g, 4.93 mL, 28.2 mmol, 2.50 eq) was sealed and stirred at 130° C. (preheated oil bath) for 3 h. LCMS showed some product at rt=1.16 min and still remaining starting material at rt=0.95 min. The brown-neat reaction mixture was sealed again and the stirring was continued at 130° C. (pre-heated oil bath) for 16 h (overnight). LCMS showed complete conversion to 2 regioisomers at rt=1.17 mind and at rt=1.23 min. The viscous-brown reaction mixture was poured warm (−50° C.) on water (˜100 mL) and ethyl acetate (˜50 mL) was used to transfer the oily mixture. The brown reaction solution was stirred for 10 min, transferred into a separating funnel and extracted twice with ethyl acetate (2×˜100 mL). The organic layers were washed with water (˜50 mL) and brine (˜50 mL). The combined organic extracts were dried over sodium sulfate, filtered off and concentrated in vacuo. The brown crude product was absorbed on ISOLUTE HM-N and purified by flash chromatography (SiO₂, 0%-40% ethyl acetate in heptane) to afford (3R)-3-[(6-chloro-5-ethyl-pyridazin-3-yl)amino]piperidine-1-carboxylic acid tert-butyl ester (1.32 g, 34%) as light yellow foam. LCMS: m/z=341.1 ([{35Cl}M+H]+), 341.1 ([{37Cl}M+H]+), ESI pos. And a light yellow foam (3R)-3-[(6-chloro-4-ethyl-pyridazin-3-yl)amino]piperidine-1-carboxylic acid tert-butyl ester (545 mg, 13%) as light yellow foam. LCMS: m/z=341.1 ([{35Cl}M+H]+), 341.1 ([{37Cl}M+H]+), ESI pos.

### Step B: (6-Chloro-5-ethyl-pyridazin-3-yl)-[(3R)-3-piperidyl]amine;hydrochloride

To a solution of aforementioned (3R)-3-[(6-chloro-5-ethyl-pyridazin-3-yl)amino]piperidine-1-carboxylic acid tert-butyl ester (Example 12, Step A) (943 mg, 2.77 mmol, 1.00 eq) in dichloromethane (10 mL) and methanol (5 mL) was added at ambient temperature 4 M HCl in 1,4-dioxane (8.30 g, 6.92 mL, 27.7 mmol, 10.0 eq) dropwise. The reaction mixture was stirred at 23° C. for 16 h. LCMS showed complete conversion to product. Evaporation of the reaction mixture gave the title product as a 1:1 hydrogen chloride (786 mg, 97.37%) as light yellow solid. LCMS: m/z=241.1 ([{35Cl}M+H]+), 243.1 ([{37Cl}M+H]+), ESI pos.

### Step C: (6-Chloro-5-ethyl-pyridazin-3-yl)-[(3R)-1-ethyl-3-piperidyl]amine

To a suspension of aforementioned (6-chloro-5-ethyl-pyridazin-3-yl)-[(3R)-3-piperidyl]amine;hydrochloride (Example 12, Step B) (400 mg, 1.44 mmol, 1.00 eq) in dichloromethane, extra dry (15 mL) was added acetaldehyde (158.9 mg, 203.7 μL, 3.61 mmol, 2.50 eq) followed by the addition of sodium acetate (295.9 mg, 3.61 mmol, 2.50 eq) under ice-bath cooling. Sodium triacetoxyborohydride (562.6 mg, 2.65 mmol, 1.80 eq) was added at 0° C. The reaction mixture was stirred at 0° C. for 5 min and at room temperature for 3 h (the reaction mixture turned brownish). LCMS showed complete conversion to product. The reaction mixture was carefully basified with NaHCO₃-solution (25 mL) and then extracted with dichloromethane (3×60 mL). The combined organic layers were dried over sodium sulfate, filtered and concentrated in vacuo. The crude product was adsorbed on ISOLUTE HM-N and purified by flash chromatography (SiO₂, gradient 0% to 100% (dichloromethane:methanol:NH₄OH 110:10:1) in dichloromethane) to afford the title compound (204 mg, 52.6% yield) as light brown oil. LCMS: m/z=269.1 ([{35Cl}M+H]+), 271.1 ([{37Cl}M+H]+), ESI pos.

### Step D: 5-Ethyl-N-[(3R)-1-ethyl-3-piperidyl]-6-(3-fluoro-1H-indol-6-yl)pyridazin-3-amine

A mixture of aforementioned (6-chloro-5-ethyl-pyridazin-3-yl)-[(3R)-1-ethyl-3-piperidyl]amine (65 mg, 241.82 μmol, 1.0 eq), 3-fluoro-6-(4,4,5,5-tetramethyl-1,3,2-dioxaborolan-2-yl)-1H-indole (Example 2, step A) (97.64 mg, 362.74 μmol, 1.5 eq), potassium carbonate (160.43 mg, 1.16 mmol, 4.8 eq) and 1,1′-bis(diphenylphosphino)ferrocene-palladium(ii) dichloride dichloromethane complex (29.62 mg, 36.27 μmol, 0.15 eq) in 1,4-dioxane (2 mL) and water (1 mL) was flushed with argon and stirred at 90° C. for 4 hours. Then, the reaction mixture was cooled to room temperature and extracted with ethyl acetate (30 mL) and water (30 mL). The aqueous layer was back-extracted with ethyl acetate (30 mL). The organic layers were washed with water (20 mL) and brine (20 mL). The combined organic layers were dried over sodium sulfate, filtered and concentrated in vacuo. The crude product was adsorbed on ISOLUTE HM-N and purified by flash chromatography (silica gel, gradient 0% to 100% (dichloromethane:methanol:NH₄OH 110:10:1) in dichloromethane) followed by crystallisation with heptane/ethyl acetate (1:1) to afford the title compound (22 mg, 24% yield) as light brown solid. LCMS m/z: 368.2 [M+H]⁺, ESI pos.

### Example 13: N-[(3R)-1-Ethyl-3-piperidyl]-6-(1H-indol-6-yl)-5-methyl-1,2,4-triazin-3-amine

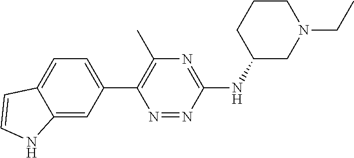 [SMILES: CCN1CCC[C@@H](Nc2nnc(-c3ccc4cc[nH]c4c3)c(C)n2)C1]

### Step A: 6-Chloro-N-[(3R)-1-ethyl-3-piperidyl]-5-methyl-1,2,4-triazin-3-amine

To a mixture of 3,6-dichloro-5-methyl-1,2,4-triazine (CAS #132434-82-3, 1.00 g, 6.1 mmol, 1.0 eq) and [(3R)-1-ethyl-3-piperidyl]amine (CAS #1020396-26-2, 1.24 g, 9.15 mmol, 1.5 eq) in 1,4-dioxane (20 mL) was added N,N-diisopropylethylamine (814 mg, 1.1 mL, 6.3 mmol, 1.03 eq). The reaction mixture was stirred at room temperature for 16 hours. The reaction mixture was extracted with dichloromethane and water. The organic layer was washed with brine. The aqueous layers were back extracted twice with dichloromethane. The combined organic layers were dried over sodium sulfate, filtered and concentrated in vacuo. The crude product was adsorbed on ISOLUTE HM-N and purified by flash chromatography (silica gel, 40 g, gradient 0% to 10% methanol in dichloromethane) to afford the title compound (1.32 g, 80% yield) as a green solid. m/z 256.3 [M+H]+, ESI pos. This step was repeated as described above to undergo the next step.

### Step B: N-[(3R)-1-Ethyl-3-piperidyl]-6-(1H-indol-6-yl)-5-methyl-1,2,4-triazin-3-amine

Aforementioned 6-chloro-N-[(3R)-1-ethyl-3-piperidyl]-5-methyl-1,2,4-triazin-3-amine (Example 13, step A) (50 mg, 196 μmol, 1.00 eq), 1H-indol-6-ylboronic acid (47 mg, 293 μmol, 1.50 eq, CAS #147621-18-9), cesium carbonate (191 mg, 587 μmol, 3.0 eq) and 1,1′-bis(diphenylphosphino)ferrocene-palladium(ii) dichloride dichloromethane complex (14 mg, 19.6 μmol, 0.1 eq) were dissolved in 1,4-dioxane (2.0 mL) and water (1.0 mL). The mixture was flushed with argon and stirred at 90° C. until LCMS showed consumption of starting material. The reaction mixture was cooled to room temperature and extracted with ethyl acetate (30 mL). The combined organic phases were washed with brine and water, dried over sodium sulfate, filtered and concentrated in vacuo. The crude product was purified by preparative HPLC (column: Gemini NX, 12 nm, 5 μm, 100×30 mm, MeCN/Water+0.1% TEA) to afford the title compound (24.6 mg, 37% yield). LCMS: m/z=337.3 [M+H]⁺, ESI pos.

### Example 14: N-[(3R)-1-Ethyl-3-piperidyl]-6-(3-fluoro-1H-indol-6-yl)-5-methyl-1,2,4-triazin-3-amine

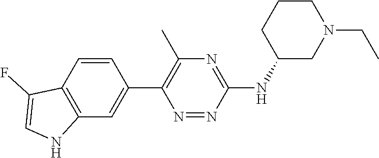 [SMILES: CCN1CCC[C@@H](Nc2nnc(-c3ccc4c(F)c[nH]c4c3)c(C)n2)C1]

A mixture of aforementioned 6-chloro-N-[(3R)-1-ethyl-3-piperidyl]-5-methyl-1,2,4-triazin-3-amine (Example 13, step A) (79 mg, 308.9 μmol, 1.00 eq), 3-fluoro-6-(4,4,5,5-tetramethyl-1,3,2-dioxaborolan-2-yl)-1H-indole (Example 2, step A) (124.7 mg, 463.3 μmol, 1.50 eq), potassium carbonate (204.9 mg, 1.48 mmol, 4.80 eq) and 1,1′-bis(diphenylphosphino)ferrocene-palladium(ii) dichloride dichloromethane complex (37.8 mg, 46.3 μmol, 0.15 eq) in 1,4-dioxane (2.38 mL) and water (1.19 mL) was flushed with argon and stirred at 90° C. overnight. LCMS showed complete conversion to product. The reaction mixture was cooled to room temperature and extracted with ethyl acetate (30 mL) and water (30 mL). The aqueous layer was back extracted with ethyl acetate (30 mL). The organic layers were washed with water (20 mL) and brine (20 mL). The combined organic layers were dried over sodium sulfate, filtered and concentrated in vacuo. The crude product was adsorbed on ISOLUTE HM-N and purified by flash chromatography (SiO₂, gradient 0% to 100% (dichloromethane:methanol:NH₄OH 110:10:1) in dichloromethane) followed by crystallisation with heptane/ethylacetate (1:1) to afford the title compound (52 mg, 45% yield) as grey solid. LCMS: m/z=355.2 [M+H]⁺, ESI pos.

### Example 15: N-[(3R)-1-Ethyl-3-piperidyl]-7-(1H-indol-6-yl)-1-methyl-pyrazolo[3,4-d]pyridazin-4-amine

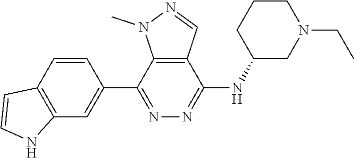 [SMILES: CCN1CCC[C@@H](Nc2nnc(-c3ccc4cc[nH]c4c3)c3c2cnn3C)C1]

### Step A: Diethyl 2-methylpyrazole-3,4-dicarboxylate

To a solution of diethyl 1H-pyrazole-4,5-dicarboxylate (4.00 g, 18.9 mmol, 1.0 eq) in MeCN (60 mL) was added potassium carbonate (5.21 g, 37.7 mmol, 2.0 eq). The mixture was stirred at 20° C. for 0.5 hrs, and then iodo methane (4.01 g, 28.3 mmol, 1.5 eq) was added to the mixture. The mixture was stirred at 20° C. for 12 hrs. Upon reaction completion (TLC: PE:EtOAc=3:1), the mixture was quenched with H₂O (100 mL) and extracted with EtOAc (100 mL*3). The organic phase was washed with brine (100 mL*2), dried over anhydrous Na₂SO₄, filtered and the filtrate was concentrated under reduce pressure. The residue was purified by column chromatography (SiO₂, petroleum ether:ethyl acetate, 5:1 to 2:1) to afford the title compound (1.35 g, 32% yield) as a colorless oil. ¹H NMR (400 MHz, DMSO-d₆) δ=7.88 (s, 1H), 4.37, 4.21 (q, 2H each), 3.94 (s, 3H), 1.34-1.21 (m, 6H).

### Step B: 1-Methylpyrazolo[3,4-d]pyridazine-4,7-diol

To a solution of diethyl 2-methylpyrazole-3,4-dicarboxylate (1.1 g, 4.86 mmol, 1.0 eq) in methanol (20 mL) was added hydrazine monohydrate (0.71 mL, 14.6 mmol, 3.0 eq). The mixture was stirred at 20° C. for 24 hrs. The reaction mixture was concentrated under reduce pressure. The residue was purified by RP flash (CombiFlash (0.1% NH₃·H₂O aqueous-MeCN condition) and following by lyophilization to afford the title compound (100 mg, 12% yield) as a white solid. LCMS: m/z 167.1 [M+H]⁺, ESI pos.

### Step C: 4,7-Dichloro-1-methyl-pyrazolo[3,4-d]pyridazine

A mixture of aforementioned 1-methylpyrazolo[3,4-d]pyridazine-4,7-diol (200 mg, 1.20 mmol, 1.0 eq) in POCl₃ (2.0 mL) was stirred at 60° C. for 12 hrs. Upon reaction completion, the mixture was concentrated under reduce pressure to afford the title compound (220 mg, 90% yield) as a yellow solid. LCMS: m/z 202.8 [M+H]+, ESI pos.

### Step D: 7-Chloro-N-[(3R)-1-ethyl-3-piperidyl]-1-methyl-pyrazolo[3,4-d]pyridazin-4-amine

To a solution of aforementioned 4,7-dichloro-1-methyl-pyrazolo[3,4-d]pyridazine (200 mg, 0.99 mmol, 1.0 eq) and (3R)-1-ethylpiperidin-3-amine (CAS #1020396-26-2, 152 mg, 1.18 mmol, 1.20 eq) in NMP (1 mL) was added potassium carbonate (272 mg, 1.97 mmol, 2.0 eq). The mixture was heated to 85° C. and stirred for 16 hrs under N₂. Upon reaction completion, the reaction mixture was cooled to 20° C. and purified by reversed phase flash (CombiFlash 0.1% TFA aqueous-ACN condition) and following by lyophilization to afford the title compound (70 mg, 24% yield) as a yellow solid. LCMS: m/z 295.0 [M+H]⁺, ESI pos. The synthesis was repeated as described above.

### Step E: N-[(3R)-1-Ethyl-3-piperidyl]-7-(1H-indol-6-yl)-1-methyl-pyrazolo[3,4-d]pyridazin-4-amine

A mixture of 7-chloro-N-[(3R)-1-ethyl-3-piperidyl]-1-methyl-pyrazolo[3,4-d]pyridazin-4-amine (100 mg, 0.34 mmol, 1.00 eq), 1H-indol-6-ylboronic acid (CAS #147621-18-9, 100 mg, 0.62 mmol, 1.83 eq), potassium carbonate (225 mg, 1.63 mmol, 4.80 eq) and 1,1′-bis(diphenylphosphino)ferrocene-palladium(ii) dichloride dichloromethane complex (42 mg, 0.05 mmol, 0.15 eq) in 1,4-dioxane (2.0 mL) and water (1.0 mL) was flushed with argon and stirred at 95° C. for 16 hours. The reaction mixture was cooled to room temperature and then extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 9:1:0.05) in dichloromethane). All fractions containing product were combined and concentrated in vacuo. The residue was adsorbed on ISOLUTE HM-N and repurified by flash chromatography (Si-amine, 12 g, gradient 0% to 5% methanol in ethyl acetate) to afford the title compound (70 mg, 52% yield) as an off-white foam. LCMS: m/z 376.3 [M+H]⁺, ESI pos.

### Example 16: N-[(3R)-1-Ethyl-3-piperidyl]-6-(5-methyl-1H-indol-6-yl)pyridazin-3-amine

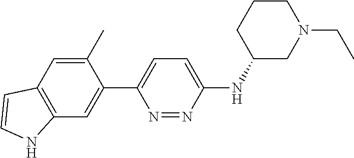 [SMILES: CCN1CCC[C@@H](Nc2ccc(-c3cc4[nH]ccc4cc3C)nn2)C1]

### Step A: 6-Chloro-N-[(3R)-1-ethyl-3-piperidyl]pyridazin-3-amine

To a mixture of 3,6-dichloropyridazine (CAS #141-30-0, 0.600 g, 4.03 mmol, 1.00 eq) and (3R)-1-ethylpiperidin-3-amine (CAS #1020396-26-2, 620 mg, 4.84 mmol, 1.20 eq) in N-methyl-2-pyrrolidinone (3.4 mL) was added N,N-diisopropylethylamine (1.33 g, 1.8 mL, 10.3 mmol, 2.56 eq). The reaction mixture was stirred at 120° C. for 16 h. The reaction mixture was cooled to room temperature and extracted with ethyl acetate and aq. 5% LiCl-solution. The aqueous layer was backextracted with ethyl acetate. The organic layers were washed twice with aq. 5% LiCl-solution, once with water and once with brine. The combined organic layers were dried over sodium sulfate, filtered and concentrated in vacuo. The crude product was adsorbed on ISOLUTE HM-N and purified by flash chromatography (silica gel, 25 g, gradient 0% to 100% (dichloromethane:methanol:NH₄OH 9:1:0.05) in dichloromethane) to afford the title compound (567 mg, 56% yield) as a light yellow solid. LCMS: m/z 241.1 [M+H]⁺, ESI pos.

### Step B: 5-Methyl-6-(4,4,5,5-tetramethyl-1,3,2-dioxaborolan-2-yl)-1H-indole

To a solution of 6-bromo-5-methyl-1H-indole (CAS #248602-16-6, 330 mg, 1.57 mmol, 1.00 eq) and bis(pinacolato)diboron (478 mg, 1.88 mmol, 1.20 eq) in 1,4-dioxane (3.7 mL) were added potassium acetate (462 mg, 4.71 mmol, 3.00 eq) followed by 1,1′-bis(diphenylphosphino)ferrocene-palladium(II)dichloride dichloromethane complex (130 mg, 0.16 mmol, 0.10 eq). The reaction mixture was flushed with argon and stirred at 90° C. for 16 hours. The reaction mixture was cooled to room temperature, adsorbed on ISOLUTE HM-N and purified by flash chromatography (silica gel, 25 g, gradient 0% to 20% ethyl acetate in heptane) to afford the title compound (182 mg, 43% yield) as a light yellow solid. LCMS: m/z 258.0 [M+H]⁺, ESI pos.

### Step C: N-[(3R)-1-Ethyl-3-piperidyl]-6-(5-methyl-1H-indol-6-yl)pyridazin-3-amine

A mixture of 6-chloro-N-[(3R)-1-ethyl-3-piperidyl]pyridazin-3-amine (Example 16, step A) (60 mg, 0.24 mmol, 1.00 eq), 5-methyl-6-(4,4,5,5-tetramethyl-1,3,2-dioxaborolan-2-yl)-1H-indole (Example 15, step B) (100 mg, 0.37 mmol, 1.56 eq), potassium carbonate (156 mg, 1.13 mmol, 4.77 eq) and 1,1′-bis(diphenylphosphino)ferrocene-palladium(II) dichloride dichloromethane complex (24 mg, 0.03 mmol, 0.12 eq) in 1,4-dioxane (1.6 mL) and water (0.80 mL) was flushed with argon and stirred at 100° C. for 4 hours. The reaction mixture was cooled to room temperature and extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 9:1:0.05) in dichloromethane). All fractions containing product were combined and concentrated in vacuo. The residue was triturated with ethyl acetate to afford the title compound (50 mg, 60% yield) as a dark brown solid. LCMS: m/z 336.3 [M+H]⁺, ESI pos.

### Example 17: 3-[[6-(3-Fluoro-1H-indol-6-yl)-5-methyl-pyridazin-3-yl]amino]-1-methyl-cyclobutanol

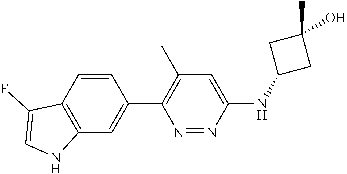 [SMILES: Cc1cc(N[C@H]2C[C@@](C)(O)C2)nnc1-c1ccc2c(F)c[nH]c2c1]

### Step A: 3-[(6-Chloro-5-methyl-pyridazin-3-yl)amino]-1-methyl-cyclobutanol and 3-[(6-chloro-4-methyl-pyridazin-3-yl)amino]-1-methyl-cyclobutanol

To a solution of 3,6-dichloro-4-methyl-pyridazine (CAS #19064-64-3, 300 mg, 1.84 mmol, 1.00 eq) in N-methyl-2-pyrrolidinone (3.0 mL) was added N,N-diisopropylethylamine (962 mg, 1.3 mL, 7.44 mmol, 4.04 eq) and cis-3-amino-1-methyl-cyclobutanol hydrochloride (CAS #1523606-23-6, 253 mg, 1.84 mmol, 1.00 eq). The mixture was stirred at 180° C. under microwave irradiation for 2 hours. The reaction mixture was extracted with ethyl acetate and aq. 10% LiCl-solution. The aqueous layer was backextracted with ethyl acetate. The organic layers were washed two times with aq. 10% LiCl-solution, once with water and once with brine. The combined organic layers were dried over sodium sulfate, filtered and concentrated in vacuo. The crude product was adsorbed on ISOLUTE HM-N and purified by flash chromatography (silica gel, 24 g, gradient 0% to 5% methanol in dichlormethane) to afford 3-[(6-chloro-5-methyl-pyridazin-3-yl)amino]-1-methyl-cyclobutanol (100 mg, 23% yield) as an off-white solid; LCMS: m/z 228.0 [M+H]⁺, ESI pos and 3-[(6-chloro-4-methyl-pyridazin-3-yl)amino]-1-methyl-cyclobutanol (70 mg, 16% yield) as an off-white solid; LCMS: m/z 228.1 [M+H]⁺, ESI pos.

### Step B: 3-[[6-(3-Fluoro-1H-indol-6-yl)-5-methyl-pyridazin-3-yl]amino]-1-methyl-cyclobutanol

A mixture of 3-[(6-chloro-5-methyl-pyridazin-3-yl)amino]-1-methyl-cyclobutanol (Example 17, step A) (60 mg, 0.25 mmol, 1.00 eq), 3-fluoro-6-(4,4,5,5-tetramethyl-1,3,2-dioxaborolan-2-yl)-1H-indole (Example 2, step A) (110 mg, 0.40 mmol, 1.60 eq), potassium carbonate (175 mg, 1.27 mmol, 5.06 eq) and 1,1′-bis(diphenylphosphino)ferrocene-palladium(II) dichloride dichloromethane complex (28 mg, 0.03 mmol, 0.14 eq) in 1,4-dioxane (1.6 mL) and water (0.80 mL) was flushed with argon and stirred at 90° C. for 16 h. The reaction mixture was cooled to room temperature and extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 9:1:0.05) in dichloromethane) to afford the title compound (52 mg, 57% yield) as a brown foam. LCMS: m/z 327.1 [M+H]⁺, ESI pos.

### Example 18A and 18B: (3aS,7aR)-1-[6-(3-Fluoro-1H-indol-6-yl)-5-methyl-pyridazin-3-yl]-6-methyl-3,3a,4,5,7,7a-hexahydro-2H-pyrrolo[2,3-c]pyridine and (3aR,7aS)-1-[6-(3-fluoro-1H-indol-6-yl)-5-methyl-pyridazin-3-yl]-6-methyl-3,3a,4,5,7,7a-hexahydro-2H-pyrrolo[2,3-c]pyridine

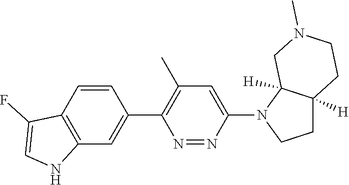 [SMILES: Cc1cc(N2CC[C@@H]3CCN(C)C[C@@H]32)nnc1-c1ccc2c(F)c[nH]c2c1]

### Step A: tert-Butyl-1-(6-chloro-5-methyl-pyridazin-3-yl)-3,3a,4,5,7,7a-hexahydro-2H-pyrrolo[2,3-c]pyridine-6-carboxylate

A mixture of 1,2,3,3a,4,5,7,7a-octahydropyrrolo[2,3-c]pyridine-6-carboxylic acid tert-butyl ester (CAS #1196147-27-9, 2.33 g, 10.31 mmol, 1.4 eq), 3,6-dichloro-4-methyl-pyridazine (CAS #19064-64-3, 1.2 g, 7.36 mmol, 1.0 eq) and N,N-diisopropylethylamine (2.57 mL, 14.7 mmol, 2.0 eq) in N-methyl-2-pyrrolidinone (3 mL) was stirred at 130° C. for 24 h. The reaction mixture was extracted with ethyl acetate (2×80 mL) and half-saturated NaHCO₃. The organic layers were washed with water (80 mL) and brine (80 mL). The combined organic layers were dried over sodium sulfate, filtered and concentrated in vacuo. The residue was purified by flash chromatography (silica gel, 0% to 50% ethyl acetate in heptane) to afford the title compound (1.49 g, 57% yield) as a light yellow solid. LCMS: m/z 353.3 ([{³⁵Cl}M+H]+), 355.3 ([{³⁷Cl}M+H]⁺), ESI pos.

### Step B: 1-(6-Chloro-5-methyl-pyridazin-3-yl)-2,3,3a,4,5,6,7,7a-octahydropyrrolo[2,3-c]pyridine hydrochloride

tert-Butyl-1-(6-chloro-5-methyl-pyridazin-3-yl)-3,3a,4,5,7,7a-hexahydro-2H-pyrrolo[2,3-c]pyridine-6-carboxylate (1.48 g, 4.19 mmol, 1.0 eq) was dissolved in dichloromethane (12.7 mL) and methanol (6.4 mL). 4 M HCl in 1,4-dioxane (9.4 mL, 37.8 mmol, 9.0 eq) was added dropwise to the reaction mixture and it was stirred at 23° C. for 3 h. The reaction mixture was concentrated in vacuo afford the title compound (1.40 g, 99% yield) as an orange solid. LCMS: m/z 253.2 [M+H]⁺, ESI pos.

### Step C: 1-(6-Chloro-5-methyl-pyridazin-3-yl)-6-methyl-3,3a,4,5,7,7a-hexahydro-2H-pyrrolo[2,3-c]pyridine

1-(6-Chloro-5-methyl-pyridazin-3-yl)-2,3,3a,4,5,6,7,7a-octahydropyrrolo[2,3-c]pyridine hydrochloride (1.4 g, 4.16 mmol, 1.0 eq) was dissolved in 1,2-dichloroethane (40.0 mL) and triethylamine (649 mg, 0.89 mL, 6.41 mmol, 1.54 eq) was added. The reaction mixture was stirred at 23° C. for 5 minutes. Then formaldehyde (37% aqueous solution, 758 mg, 0.70 mL, 9.33 mmol, 2.24 eq) was added followed by the portion-wise addition of sodium triacetoxyborohydride (CAS #56553-60-7, 3.53 g, 16.7 mmol, 4.0 eq). The reaction mixture was stirred at room temperature for 1 h. Afterwards, the reaction mixture was carefully quenched with saturated aq. NaHCO₃-solution and extracted with dichloromethane (5×30 mL). The combined organic layers were dried over sodium sulfate, filtered and concentrated in vacuo. The crude product was purified by flash chromatography (silica gel, gradient 0% to 5% methanol in dichloromethane) to afford the title compound (1.2 g, 97% yield) as a light yellow solid. LCMS m/z 267.2 [M+H]⁺, ESI pos.

### Step D: (3aS,7aR)-1-[6-(3-fluoro-1H-indol-6-yl)-5-methyl-pyridazin-3-yl]-6-methyl-3,3a,4,5,7,7a-hexahydro-2H-pyrrolo[2,3-c]pyridine (A) and (3aR,7aS)-1-[6-(3-fluoro-1H-indol-6-yl)-5-methyl-pyridazin-3-yl]-6-methyl-3,3a,4,5,7,7a-hexahydro-2H-pyrrolo[2,3-c]pyridine (B)

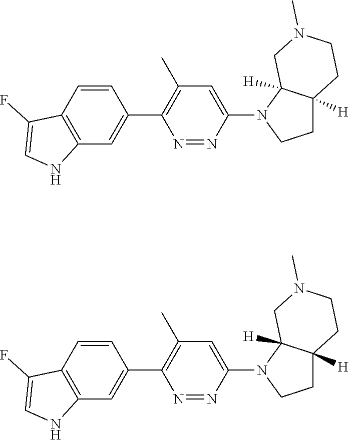 [SMILES: Cc1cc(N2CC[C@@H]3CCN(C)C[C@@H]32)nnc1-c1ccc2c(F)c[nH]c2c1 ; Cc1cc(N2CC[C@H]3CCN(C)C[C@H]32)nnc1-c1ccc2c(F)c[nH]c2c1]

A mixture of aforementioned 1-(6-chloro-5-methyl-pyridazin-3-yl)-6-methyl-3,3a,4,5,7,7a-hexahydro-2H-pyrrolo[2,3-c]pyridine (84 mg, 0.30 mmol, 1.0 eq), 3-fluoro-6-(4,4,5,5-tetramethyl-1,3,2-dioxaborolan-2-yl)-1H-indole (113 mg, 0.42 mmol, 1.40 eq), potassium carbonate (198 mg, 1.44 mmol, 4.80 eq) and 1,1′-bis(diphenylphosphino)ferrocene-palladium(ii) dichloride dichloromethane complex (CAS #95464-05-4, 37 mg, 0.045 mmol, 0.15 eq) in 1,4-dioxane (2 mL) and water (1 mL) was flushed with argon and stirred at 90° C. for 6 h. The reaction mixture was cooled to room temperature and extracted with ethyl acetate (30 mL) and water (30 mL). The aqueous layer was back extracted with ethyl acetate (30 mL). The organic layers were washed with water (20 mL) and brine (20 mL). The combined organic layers were dried over sodium sulfate, filtered and concentrated in vacuo. The product was purified by flash chromatography (silica gel, gradient 0% to 100% (dichloromethane:methanol:NH₄OH 110:10:1) in dichloromethane) to afford the title compound (84 mg, 73% yield) as a light brown solid. Chiral separation was done by SFC (Column chiral AD-H, 5 μm, 250×20 mm; elution: 35% methanol in CO₂) to yield the first eluting title compound A (28 mg, 34% yield) as a light brown solid and the second eluting title compound B (32 mg, 39% yield) as a light brown solid. LCMS: m/z 366.3 [M+H]⁺, ESI pos.

### Example A

A compound of formula Ib can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B

A compound of formula Ib can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula Ib or Ib′ 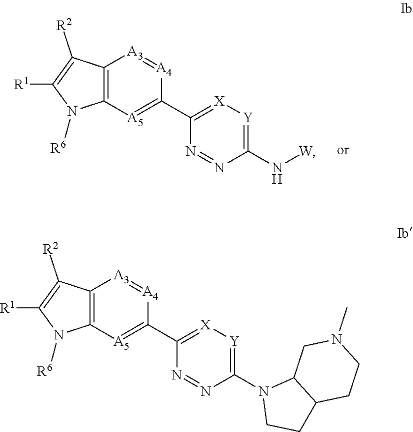
wherein
A₃ is selected from N or CR³;
A₄ is selected from N or CR⁴;
A₅ is selected from N or CR⁵;
Y is selected from N or CH and X is CR⁷,
or X and Y, and the atoms to which they are attached, form a 5-member heteroaryl ring comprising 2 N heteroatoms, wherein one N heteroatom is substituted with either H or alkyl;
R¹ is selected from alkyl, cyano, or H;
R² is selected from alkyl, cyano, H or halo;
R³ is H;
R⁴ is selected from H, alkyl, alkoxy or halo;
R⁵ is selected from H or halo;
R⁶ is H;
R⁷ is alkyl or haloalkyl;
W is optionally substituted cycloalkyl or optionally substituted heterocycle ring comprising a single N heteroatom, wherein optionally substituted cycloalkyl can be substituted with 1 or 2 substituents independently selected from H, OH or alkyl, and optionally substituted heterocycle ring comprising a single N heteroatom can be substituted with alkyl or haloalkyl;
and pharmaceutically acceptable salts thereof.

2. A compound of formula Ib 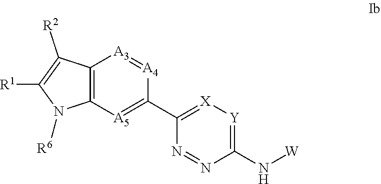
wherein
A₃ is selected from N or CR³;
A₄ is selected from N or CR⁴;
A₅ is selected from N or CR⁵;
Y is selected from N or CH and X is CR⁷,
or X and Y, and the atoms to which they are attached, form a 5-member heteroaryl ring comprising 2 N heteroatoms, wherein one N heteroatom is substituted with either H or alkyl;
R¹ is selected from alkyl, cyano, or H;
R² is selected from alkyl, cyano, H or halo;
R³ is H;
R⁴ is selected from H, alkyl, alkoxy or halo;
R⁵ is selected from H or halo;
R⁶ is H;
R⁷ is alkyl or haloalkyl;
W is optionally substituted cycloalkyl or optionally substituted heterocycle ring comprising a single N heteroatom, wherein optionally substituted cycloalkyl can be substituted with 1 or 2 substituents independently selected from H, OH or alkyl, and optionally substituted heterocycle ring comprising a single N heteroatom can be substituted with alkyl or haloalkyl;
and pharmaceutically acceptable salts thereof.

3. A compound according to claim 1, wherein Y is selected from N or CH and X is CR⁷.

4. A compound according to claim 1, wherein R¹ is H.

5. A compound according to claim 1, wherein R² is H or halo.

6. A compound according to claim 1, wherein A₃ is CR³.

7. A compound according to claim 1, wherein A₄ is CR⁴.

8. A compound according to claim 1, wherein A₅ is CR⁵.

9. A compound according to claim 1, wherein R⁴ is selected from H or halo.

10. A compound according to claim 1, wherein R⁴ is H.

11. A compound according to claim 1, wherein R⁵ is H.

12. A compound according to claim 1, wherein R⁷ is alkyl.

13. A compound according to claim 1, wherein W is a piperidine ring substituted with alkyl.

14. A compound according to claim 1, wherein
A₃ is CR³;
A₄ is CR⁴;
A₅ is CR⁵;
Y is selected from N or CH and X is CR⁷;
R¹ is alkyl, cyano, or H;
R² is alkyl, cyano, H or halo;
R³ is H;
R⁴ is H or halo;
R⁵ is H or halo;
R⁶ is H;
R⁷ is alkyl or haloalkyl;
W is a piperidine ring substituted with alkyl;
and pharmaceutically acceptable salts thereof.

15. A compound according to claim 1, wherein
A₃ is CR³;
A₄ is CR⁴;
A₅ is CR⁵;
Y is selected from N or CH and X is CR⁷;
R¹ is H;
R² is H or halo;
R³ is H;
R⁴ is H;
R⁵ is H;
R⁶ is H;
R⁷ is alkyl or haloalkyl;
W is a piperidine ring substituted with alkyl;
and pharmaceutically acceptable salts thereof.

16. A compound according to claim 1, wherein
A₃ is CR³;
A₄ is CR⁴;
A₅ is CR⁵;
Y is selected from N or CH and X is CR⁷;
R¹ is H;
R² is H or halo;
R³ is H;
R⁴ is H;
R⁵ is H;
R⁶ is H;
R⁷ is alkyl;
W is a piperidine ring substituted with alkyl;
and pharmaceutically acceptable salts thereof.

17. A compound according to claim 1, wherein the compound is selected from
N-[(3R)-1-Ethyl-3-piperidyl]-6-(1H-indol-6-yl)-5-methyl-pyridazin-3-amine;
N-[(3R)-1-Ethyl-3-piperidyl]-6-(3-fluoro-1H-indol-6-yl)-5-methyl-pyridazin-3-amine;
N-[(3R)-1-Ethyl-3-piperidyl]-6-(5-fluoro-1H-indol-6-yl)-5-methyl-pyridazin-3-amine;
N-[(3R)-1-Ethyl-3-piperidyl]-5-methyl-6-(3-methyl-1H-indol-6-yl)pyridazin-3-amine;
6-(3-Chloro-1H-indol-6-yl)-N-[(3R)-1-ethyl-3-piperidyl]-5-methyl-pyridazin-3-amine;
N-[(3R)-1-Ethyl-3-piperidyl]-5-methyl-6-(2-methyl-1H-indol-6-yl)pyridazin-3-amine;
6-[6-[[(3R)-1-Ethyl-3-piperidyl]amino]-4-methyl-pyridazin-3-yl]-1H-indole-3-carbonitrile;
6-[6-[[(3R)-1-Ethyl-3-piperidyl]amino]-4-methyl-pyridazin-3-yl]-1H-indole-2-carbonitrile;
6-(3-Fluoro-1H-indol-6-yl)-5-methyl-N-[(3R)-1-methyl-3-piperidyl]pyridazin-3-amine;
N-[(3R)-1-Ethyl-3-piperidyl]-6-(1H-indol-6-yl)-5-methyl-pyridazin-3-amine;
N-[(3R)-1-Ethyl-3-piperidyl]-6-(3-fluoro-1H-indol-6-yl)-5-(trifluoromethyl)pyridazin-3-amine;
5-Ethyl-N-[(3R)-1-ethyl-3-piperidyl]-6-(3-fluoro-1H-indol-6-yl)pyridazin-3-amine;
N-[(3R)-1-Ethyl-3-piperidyl]-6-(1H-indol-6-yl)-5-methyl-1,2,4-triazin-3-amine;
N-[(3R)-1-Ethyl-3-piperidyl]-6-(3-fluoro-1H-indol-6-yl)-5-methyl-1,2,4-triazin-3-amine;
and pharmaceutically acceptable salts thereof.

18. A compound according to claim 1, wherein the compound is selected from
N-[(3R)-1-Ethyl-3-piperidyl]-6-(1H-indol-6-yl)-5-methyl-pyridazin-3-amine;
N-[(3R)-1-Ethyl-3-piperidyl]-6-(3-fluoro-1H-indol-6-yl)-5-methyl-pyridazin-3-amine;
6-(3-Fluoro-1H-indol-6-yl)-5-methyl-N-[(3R)-1-methyl-3-piperidyl]pyridazin-3-amine;
N-[(3R)-1-Ethyl-3-piperidyl]-6-(3-fluoro-1H-indol-6-yl)-5-(trifluoromethyl)pyridazin-3-amine;
N-[(3R)-1-Ethyl-3-piperidyl]-6-(1H-indol-6-yl)-5-methyl-1,2,4-triazin-3-amine;
N-[(3R)-1-Ethyl-3-piperidyl]-6-(3-fluoro-1H-indol-6-yl)-5-methyl-1,2,4-triazin-3-amine;
and pharmaceutically acceptable salts thereof.

19. A compound according to claim 1, wherein the compound is selected from
N-[(3R)-1-Ethyl-3-piperidyl]-6-(3-fluoro-1H-indol-6-yl)-5-methyl-pyridazin-3-amine;
N-[(3R)-1-Ethyl-3-piperidyl]-6-(1H-indol-6-yl)-5-methyl-1,2,4-triazin-3-amine;
N-[(3R)-1-Ethyl-3-piperidyl]-6-(3-fluoro-1H-indol-6-yl)-5-methyl-1,2,4-triazin-3-amine;
and pharmaceutically acceptable salts thereof.

20. A compound according to claim 1, wherein the compound is N-[(3R)-1-Ethyl-3-piperidyl]-7-(1H-indol-6-yl)-1-methyl-pyrazolo[3,4-d]pyridazin-4-amine and pharmaceutically acceptable salts thereof.

21. A compound according to any claim 1, wherein the compound is selected from
N-[(3R)-1-Ethyl-3-piperidyl]-6-(5-methyl-1H-indol-6-yl)pyridazin-3-amine;
3-[[6-(3-Fluoro-1H-indol-6-yl)-5-methyl-pyridazin-3-yl]amino]-1-methyl-cyclobutanol;
and pharmaceutically acceptable salts thereof.

22. A compound according to claim 1, wherein the compound is selected from
(3aS,7aR)-1-[6-(3-Fluoro-1H-indol-6-yl)-5-methyl-pyridazin-3-yl]-6-methyl-3,3a,4,5,7,7a-hexahydro-2H-pyrrolo[2,3-c]pyridine;
(3aR,7aS)-1-[6-(3-fluoro-1H-indol-6-yl)-5-methyl-pyridazin-3-yl]-6-methyl-3,3a,4,5,7,7a-hexahydro-2H-pyrrolo[2,3-c]pyridine;
and pharmaceutically acceptable salts thereof.

23. (canceled)

24. (canceled)

25. A pharmaceutical composition comprising a compound according to claim 1 and a therapeutically inert carrier.

26. (canceled)

27. (canceled)

28. (canceled)

29. (canceled)

30. (canceled)

31. (canceled)

32. (canceled)

33. A method of inhibiting NLRP3, which method comprises administering an effective amount of a compound as claimed in claim 1 to inhibit NLRP3.

34. A method for the treatment or prophylaxis of a disease, disorder or condition, which method comprises administering an effective amount of a compound according to claim 1, wherein the disease, disorder or condition is selected from Asthma or COPD.

35. A method for the treatment or prophylaxis of a disease, disorder or condition, which method comprises administering an effective amount of a compound according to claim 1, wherein the disease, disorder or condition is selected from Parkinson's Disease or Alzheimer's Disease.

36. (canceled)
